(19) United States
(12) Patent Application Publication
(10) Pub. No.: US 2006/0228317 A1
(43) Pub. Date: Oct. 12, 2006

(54) Graft polymers and use thereof in cosmetic formulations

(75) Inventors: Lysander Chrisstoffels, Limburgerhof (DE); Peter Hossel, Schifferstadt (DE); Claudia Wood, Weinheim (DE); Maximilian Angel, Schifferstadt (DE); Klemens Mathauer, Heidelberg (DE)

Correspondence Address: CONNOLLY BOVE LODGE & HUTZ, LLP, P O BOX 2207, WILMINGTON, DE, 19899, US

(73) Assignee: BASFAKTIENGLESELLSCHAFT, Ludwigshafen (DE)

(21) Appl. No.: 10/548,867

(22) PCT Filed: Mar. 5, 2004
(86) PCT No.: PCT/EP04/02243

§ 371 Date: Sep. 14, 2005

(30) Foreign Application Priority Data
Mar. 14, 2003 (DE) 103 11 616.8

(51) Int. Cl. A61K 8/81

(52) U.S. Cl. 424/70.7; 424/70.15; 525/73

(57) ABSTRACT

The invention relates to graft polymers obtainable by free-radical graft polymerization of a) at least one N-vinyl-containing monomer b) optionally one or more further copolymerizable monomers onto a polymeric graft base c), which comprises at least one compound from the group c1) and at least one compound from the group c2), where c1) represents polyether-containing compounds c2) represents polymers which comprise at least 5% by weight of vinylpyrrolidone units d) optionally at least one crosslinker. and to their use in cosmetic preparations.

The invention relates to graft polymers and to their use as a constituent in cosmetic compositions. The graft polymers are produced by grafting monoethylenically unsaturated, open-chain N-vinylamide unit-comprising monomers onto a polymeric graft base which consists of at least 2 compounds.

Polymers are used widely in cosmetics and medicine. In soaps, creams and lotions, for example, they usually serve as formulation agents, e.g. as thickeners, foam stabilizers or water absorbents, or else for alleviating the irritative action of other ingredients, or for improving the dermal application of active ingredients. By contrast, their task in hair cosmetics is to influence the properties of the hair.

For example, conditioners are used for improving the dry and wet combability, feel, shine and appearance, and for imparting antistatic properties to the hair. Preference is given to using water-soluble polymers with polar, frequently cationic functionalities which have a greater affinity to the surface of the hair, which is negative as a result of its structure. The structure and mode of action of various hair-treatment polymers are described in Cosmetic & Toiletries 103 (1988) 23. Commercially available conditioning polymers are, for example, cationic hydroxyethylcellulose, cationic polymers based on N-vinylpyrrolidone, e.g. copolymers of N-vinylpyrrolidone and quaternized N-vinylimidazole, acrylamide and diallyldimethylammonium chloride or silicones.

For setting hairstyles, use is made of vinyllactam homopolymers and copolymers and polymers containing carboxylate groups. Requirements for hair-setting resins are, for example, a strong hold at high atmospheric humidity, elasticity, wash-off from the hair, compatibility in the formulation and a pleasant feel of the hair.

The combination of different properties, such as, for example, strong hold and pleasant feel of the hair, often presents difficulties.

WO-A-96/03969 describes haircare compositions comprising an N-vinylformamide homopolymer or a copolymer of N-vinylformamide units and a further vinyl monomer chosen from styrenes, alkyl esters of acrylic and methacrylic acid, vinyl esters of the formula CH₂═CH—OCO-alkyl, N-alkyl-substituted acrylamides and methacrylamides, esters of fumaric, itaconic and maleic acid, vinyl ethers, hydroxy-functionalized acrylates and methacrylates, acrylamide, non-alkyl-substituted acrylamides and cyclic amides. A specific example of a cyclic amide is N-vinylpyrrolidone. Further examples of vinyl monomers are secondary, tertiary and quaternary amines, such as dimethyldiallylammonium chloride, dimethylaminoethyl methacrylate or dimethylaminopropyl methacrylate.

DE 19640363 describes copolymers of N-vinylformamide and quaternized N-vinylimidazole and the uses thereof in cosmetics.

DE 19907587.5 describes the use of polymers obtainable by free-radical polymerization of at least one vinyl ester in the presence of polyether-containing compounds and optionally one or more copolymerizable monomers, and subsequent at least partial hydrolysis of the ester function in hair cosmetic formulations. A copolymerizable monomer is, inter alia, vinylformamide.

DE-A1-44 09 903 describes graft polymers comprising N-vinyl units, processes for their preparation and their use. Here, monoethylenically unsaturated monomers are grafted onto a graft base which is a polymer which in each case comprises at least 5% by weight of units of the formulae 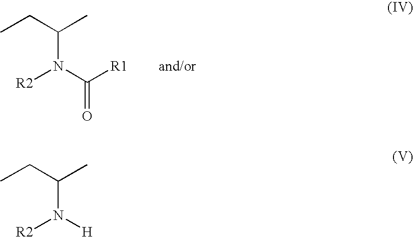
where R¹, R²═H or C₁-C₆-alkyl. Suitable monoethylenically unsaturated monomers are all ethylenically unsaturated monomers whose polymerization is not inhibited by the amine groups in free or in salt form, such as, for example, monoethylenically unsaturated mono- and dicarboxylic acids, their salts and esters with C₁-C₃₀-alcohols. Suitability of these graft copolymers as active ingredient in cosmetic formulations is not mentioned.

WO 96/34903 describes graft polymers comprising N-vinyl units, processes for their preparation and their use. Here, monoethylenically unsaturated monomers are grafted onto a graft base which is a polymer which comprises at least 3 units of a C₂-C₄-alkylene oxide, and/or polytetrahydrofuran, and then at least partially hydrolyzed. Suitability of these graft copolymers as active ingredient in cosmetic formulations is not mentioned.

U.S. Pat. No. 5,334,287 discloses graft polymers obtainable by free-radical-initiated polymerization of N-vinylcarboxamides, preferably N-vinylformamide, and optionally other monomers in the presence of monosaccharides, oligosaccharides, polysaccharides or derivatives thereof in each case, and optionally hydrolysis of the copolymerized N-vinylcarboxamide group to form vinylamine units. Suitability of these graft copolymers as active ingredient in cosmetic formulations is not mentioned.

In WO 9825981, amphiphilic graft polymers are synthesized by grafting hydrophobic monomers, such as, for example, styrene, onto polymers which comprise structural elements of the formula (IV) and/or (V). The graft polymers obtained are used inter alia as additives in cosmetic formulations.

DE-A1-196 40 363 claims the use of water-soluble copolymers as active ingredient in cosmetic formulations. As a characteristic structural element, the copolymer comprises units of the formula (VI) 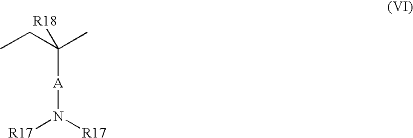
in which A is a chemical bond or an alkylene group, the radicals R¹⁷, independently of one another, are H, alkyl, cycloalkyl, aryl or aralkyl, and R¹⁸ is H, alkyl or aralkyl.

Bodycare creams which comprise a monoaldehyde-modified vinylamine polymer are known from U.S. Pat. No. 5,270,379.

Copolymers which are used, for example, as hair-setting agents and are built up from N-vinylamide monomers of the formula 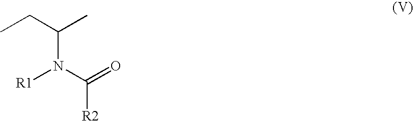
in which R1 and R2 are H or C₁-C₅-alkyl, and the comonomer is chosen from vinyl ethers, vinyllactams, vinyl halides, vinyl esters of monobasic saturated carboxylic acids, (meth)acrylic esters, amides and nitriles and esters, anhydrides and imides of maleic acid are known from DE 14 95 692.

U.S. Pat. No. 4,713,236 describes hair conditioners based on polymers comprising vinylamine units. Particular mention is made here of polyvinylamine and salts thereof, α-substituted polyvinylamines, such as, for example, poly(a-aminoacrylic acid) and also copolymers which, in addition to vinylamine, comprise, in copolymerized form, comonomers such as vinyl alcohol, acrylic acid, acrylamide, maleic anhydride, vinyl sulfonate and 2-acrylamido-2-methylpropanesulfonic acid.

WO 02/15854 A1 describes the use of hydrophilic graft copolymers with N-vinylamine and/or open-chain N-vinylamine units in cosmetic formulations. Graft polymers which are formed by grafting onto a polymeric graft base which consists of at least 2 compounds are not described.

It is an object of the present invention to find polymers which are highly suitable for cosmetic applications and which, for example in the field of hair cosmetics, have good applications-related properties, such as pleasant feel, and at the same time a good conditioning action and a good setting action.

Despite extensive efforts, there still remains a need for improvement with polymers for producing elastic hairstyles with simultaneously strong hold, even at high atmospheric humidity, good ability to be washed off and good feel of the hair. The need for improvement likewise consists, in the case of polymers for producing readily combable, detanglable hair and for the conditioning of skin and hair, in their sensorily perceptible properties, such as feel, volume, manageability etc. Also desirable are clear aqueous preparations of these polymers which are accordingly characterized by good compatibility with other formulation constituents.

In addition, there is a need for polymers which are suitable as conditioning agents for cosmetic preparations and which can be prepared with a high solids content. Of particular interest are polymers which have a high solids content, have a low viscosity whilst at the same time retaining the applications-related properties (such as, for example, combability).

We have found that this object is achieved by graft polymers obtainable by free-radical graft polymerization of
a) at least one open-chain N-vinylamide compound of the formula (I) 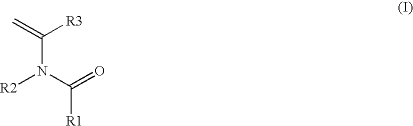
where R¹, R², R³═H or C₁— to C₆-alkyl, and
b) optionally one or more further copolymerizable monomers onto a polymeric graft base c), which comprises at least one compound from the group c1) and at least one compound from the group c2), where
c1) represents polyether-containing compounds
c2) represents polymers which comprise at least 5% by weight of vinylpyrrolidone units,
d) optionally at least one crosslinker.

Depending on the degree of grafting, the polymers used according to the invention are understood as meaning either pure graft polymers, or else mixtures of the abovementioned graft polymers with ungrafted compounds c1) and c2) and homopolymers or copolymers of the monomers a) and b).

In a preferred embodiment, the graft polymers are water-soluble or water-dispersible.

Water-soluble polymers are understood here as meaning polymers which dissolve in water at 20° C. in an amount of at least 1 g/l. Water-dispersible polymers are understood here as meaning polymers which disintegrate into dispersible particles with stirring.

Monomer a)

N-vinylamides and/or N-vinyllactams, for example, are suitable as N-vinyl-containing monomer a).

Suitable as N-vinyl-containing monomer a) are, for example, N-vinylamides of the formula (I) 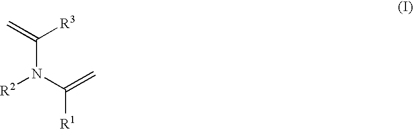
where R¹, R², R³═H or C₁— to C₆-alkyl.

For the preparation of the polymers used according to the invention, the following monomers are, for example, used as open-chain N-vinylamide compound a) of the formula (I): N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Further suitable N-vinyl-containing monomers a) are N-vinyllactams of the formula (II) 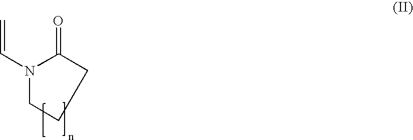
where n=1, 2, 3.

Examples of monomers of the formula (II) are N-vinylpyrrolidone (n=1) and N-vinylcaprolactam (n=3).

Further suitable N-vinyl-containing monomers a) are N-vinylpiperidone, N-vinyloxazolidone and N-vinyltriazole.

In a preferred embodiment of the invention, an N-vinylamide, in particular N-vinylformamide, is used as monomer a).

It is of course also possible to copolymerize mixtures of the respective monomers from the group a) such as, for example, mixtures of N-vinylformamide and N-vinylacetamide.

For the preparation of the graft polymers according to the invention, the following monomers are, for example, used as open-chain N-vinylamide compound a) of the formula (I): N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide. From this group of monomers, preference is given to using N-vinylformamide.

Monomer b)

As well as comprising the monomers a) and the graft base c), the graft polymers can comprise one or more further monomers b). The preferred additionally used copolymerizable monomers b) can be described by the following formula:
X—C(O)CR²⁰═CHR¹⁹
where

X is chosen from the group of radicals —OH, —OM, —OR²¹, NH₂, —NHR²¹, N(R²¹)₂;

M is a cation chosen from the group consisting of: Na⁺, K⁺, Mg⁺⁺, Ca⁺⁺, Zn⁺⁺, NH₄⁺, alkylammonium, dialkylammonium, trialkylammonium and tetraalkylammonium;

the radicals R²¹ may be identical or different and are chosen from the group consisting of —H, C₁-C₄₀ linear- or branched-chain alkyl radicals, N,N-dimethylaminoethyl, 2-hydroxyethyl, 2-methoxyethyl, 2-ethoxyethyl, hydroxypropyl, methoxypropyl or ethoxypropyl.

R²⁰ and R¹⁹ are, independently of one another, chosen from the group consisting of: —H, C₁-C₈ linear- or branched-chain alkyl chains, methoxy, ethoxy, 2-hydroxyethoxy, 2-methoxyethoxy and 2-ethoxyethyl.

Representative but nonlimiting examples of suitable monomers b) are, for example, acrylic acid or methacrylic acids or salts, esters and amides thereof. The salts can be derived from any desired nontoxic metal, ammonium or substituted ammonium counterion.

The esters can be derived from C₁-C₄₀ linear, C₃-C₄₀ branched-chain or C₃-C₄₀ carbocyclic alcohols, from polyfunctional alcohols having 2 to about 8 hydroxyl groups, such as ethylene glycol, hexylene glycol, glycerol and 1,2,6-hexanetriol, from amino alcohols or from alcohol ethers, such as methoxyethanol and ethoxyethanol, (alkyl)polyethylene glycols, (alkyl)polypropylene glycols and ethoxylated fatty alcohols, for example C₁₂-C₂₄-fatty alcohols reacted with 1 to 200 ethylene oxide units.

Also suitable are N,N-dialkylaminoalkyl acrylates and methacrylates and N-dialkylaminoalkylacrylamides and -methacrylamides of the formula (VII) 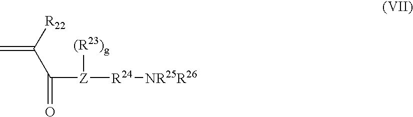
where
R²²═H, alkyl having 1 to 8 carbon atoms,
R²³═H, methyl,
R²⁴=alkylene having 1 to 24 carbon atoms, optionally substituted by alkyl,
R²⁵, R²⁶═C₁-C₄₀-alkyl radical,
Z=nitrogen when g=1 or oxygen when g=0

The amides can be unsubstituted, N-alkyl- or N-alkylamino-monosubstituted or N,N-dialkyl-substituted or N,N-dialkylamino-disubstituted, in which the alkyl or alkylamino groups are derived from C₁-C₄₀ linear, C₃-C₄₀ branched-chain, or C₃-C₄₀ carbocyclic units. Additionally, the alkylamino groups may be quaternized.

Preferred comonomers of the formula VII are N,N-dimethylaminomethyl(meth)acrylate, N,N-diethylaminomethyl(meth)acrylate, N,N-dimethylaminoethyl(meth)acrylate, N,N-diethylaminoethyl(meth)acrylate, N-[3-(dimethylamino)propyl]methacrylamide and N-[3-(dimethylamino)propyl]acrylamide.

Comonomers b) which can likewise be used are substituted acrylic acids, and salts, esters and amides thereof, where the substituents on the carbon atoms are in the two or three position of the acrylic acid, and, independently of one another, are chosen from the group consisting of C₁-C₄ alkyl, —CN, COOH, particularly preferably methacrylic acid, ethacrylic acid and 3-cyanoacrylic acid. These salts, esters and amides of these substituted acrylic acids can be chosen as described above for the salts, esters and amides of acrylic acid.

Other suitable comonomers b) are allyl esters of C₁-C₄₀ linear, C₃-C₄₀ branched-chain or C₃-C₄₀ carbocyclic carboxylic acids, vinyl or allyl halides, preferably vinyl chloride and allyl chloride, vinyl ethers, preferably methyl, ethyl, butyl or dodecyl vinyl ethers, vinyllactams, preferably vinylpyrrolidone and vinylcaprolactam, vinyl- or allyl-substituted heterocyclic compounds, preferably vinylpyridine, vinyloxazoline and allylpyridine.

Also suitable are N-vinylimidazoles of the formula VIII in which R²⁷ to R²⁹, independently of one another, are hydrogen, C₁-C₄-alkyl or phenyl:

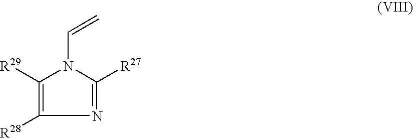

Further suitable comonomers b) are diallylamines of the formula (IX) 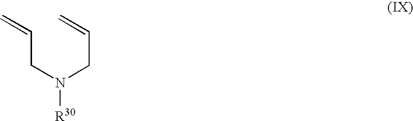
where R³⁰═C₁— to C₂₄-alkyl.

Further suitable comonomers b) are vinylidene chloride; and hydrocarbons with at least one carbon-carbon double bond, preferably styrene, alpha-methylstyrene, tert-butylstyrene, butadiene, isoprene, cyclohexadiene, ethylene, propylene, 1-butene, 2-butene, isobutylene, vinyltoluene, and mixtures of these monomers.

Particularly suitable comonomers b) are acrylic acid, methacrylic acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, n-butyl ethacrylate, isobutyl ethacrylate, t-butyl ethacrylate, 2-ethylhexyl ethacrylate, decyl ethacrylate, stearyl (meth)acrylate, 2,3-dihydroxypropyl acrylate, 2,3-dihydroxypropyl methacrylate, 2-hydroxyethyl acrylate, hydroxypropyl acrylates, 2-hydroxyethyl methacrylate, 2-hydroxyethyl ethacrylate, 2-methoxyethyl acrylate, 2-methoxyethyl methacrylate, 2-methoxyethyl ethacrylate, 2-ethoxyethyl methacrylate, 2-ethoxyethyl ethacrylate, hydroxypropyl methacrylates, glyceryl monoacrylate, glyceryl monomethacrylate, polyalkylene glycol (meth)acrylates, unsaturated sulfonic acids, such as, for example, acrylamidopropanesulfonic acid;

acrylamide, methacrylamide, ethacrylamide, N-methylacrylamide, N,N-dimethylacrylamide, N-ethylacrylamide, N-isopropylacrylamide, N-butylacrylamide, N-t-butylacrylamide, N-octylacrylamide, N-t-octylacrylamide, N-octadecylacrylamide, N-phenylacrylamide, N-methylmethacrylamide, N-ethylmethacrylamide, N-dodecylmethacrylamide, 1-vinylimidazole, 1-vinyl-2-methylvinylimidazole, N,N-dimethylaminomethyl(meth)acrylate, N,N-diethylaminomethyl(meth)acrylate, N,N-dimethylaminoethyl(meth)acrylate, N,N-diethylaminoethyl(meth)acrylate, N,N-dimethylaminobutyl(meth)acrylate, N,N-diethylaminobutyl(meth)acrylate, N,N-dimethylaminohexyl(meth)acrylate, N,N-dimethylaminooctyl(meth)acrylate, N,N-dimethylaminododecyl(meth)acrylate, N-[3-(dimethylamino)propyl]methacrylamide, N-[3-(dimethylamino)propyl]acrylamide, N-[3-(dimethylamino)butyl]methacrylamide, N-[8-(dimethylamino)octyl]methacrylamide, N-[12-(dimethylamino)dodecyl]-methacrylamide, N-[3-(diethylamino)propyl]methacrylamide, N-[3-(diethylamino)propyl]acrylamide;

maleic acid, fumaric acid, maleic anhydride and its monoesters, crotonic acid, itaconic acid, diallyldimethylammonium chloride, vinyl ethers (for example: methyl, ethyl, butyl or dodecyl vinyl ethers), methyl vinyl ketone, maleimide, vinylpyridine, vinylimidazole, vinylfuran, styrene, styrene sulfonate, allyl alcohol, and mixtures thereof.

Of these, particular preference is given to acrylic acid, methacrylic acid, maleic acid, fumaric acid, crotonic acid, maleic anhydride and monoesters thereof, methyl acrylate, methyl methacrylate, ethyl acrylate, ethyl methacrylate, n-butyl acrylate, n-butyl methacrylate, t-butyl acrylate, t-butyl methacrylate, isobutyl acrylate, isobutyl methacrylate, 2-ethylhexyl acrylate, stearyl acrylate, stearyl methacrylate, N-t-butylacrylamide, N-octylacrylamide, 2-hydroxyethyl acrylate, hydroxypropyl acrylates, 2-hydroxyethyl methacrylate, hydroxypropyl methacrylates, alkylene glycol(meth)acrylates, styrene, unsaturated sulfonic acids, such as, for example, acrylamidopropanesulfonic acid, vinylpyrrolidone, vinylcaprolactam, vinyl ethers (e.g.: methyl, ethyl, butyl or dodecyl vinyl ethers), 1-vinylimidazole, 1-vinyl-2-methylimidazole, N,N-dimethylaminomethyl methacrylate and N-[3-(dimethylamino)propyl]methacrylamide; 3-methyl-1-vinylimidazolium chloride, 3-methyl-1-vinylimidazolium methylsulfate, N,N-dimethylaminoethyl methacrylate, N-[3-(dimethylamino)propyl]methacrylamide quaternized with methyl chloride, methyl sulfate or diethyl sulfate.

Monomers containing a basic nitrogen atom can be quaternized here in the following manner:

For the quaternization of the amines, alkyl halides having 1 to 24 carbon atoms in the alkyl group are, for example, suitable, e.g. methyl chloride, methyl bromide, methyl iodide, ethyl chloride, ethyl bromide, propyl chloride, hexyl chloride, dodecyl chloride, lauryl chloride and benzyl halides, in particular benzyl chloride and benzyl bromide. Further suitable quaternizing agents are dialkyl sulfates, in particular dimethyl sulfate or diethyl sulfate. The quaternization of the basic amines can also be carried out with alkylene oxides, such as ethylene oxide or propylene oxide, in the presence of acids. Preferred quaternizing agents are, methyl chloride, dimethyl sulfate or diethyl sulfate.

The quaternization can be carried out before the polymerization or after the polymerization.

Furthermore, the reaction products of unsaturated acids, such as, for example acrylic acid or methacrylic acid, with a quaternized epichlorohydrin of the formula (X)(R³¹═C₁— to C₄₀-alkyl) can be used.

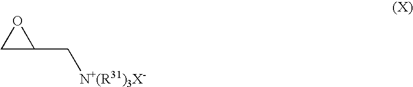

Examples thereof are:

(meth)acryloyloxyhydroxypropyltrimethylammonium chloride and (meth)acryloyloxyhydroxypropyltriethylammonium chloride.

The basic monomers can also be cationized by neutralizing them with mineral acids, such as, for example, sulfuric acid, hydrochloric acid, hydrobromic acid, hydroiodic acid, phosphoric acid or nitric acid, or with organic acids, such as, for example, formic acid, acetic acid, lactic acid or citric acid.

In addition to the abovementioned monomers, it is also possible to use, as comonomers b), so-called macromonomers, such as, for example, silicone-containing macromonomers with one or more free-radically polymerizable groups, or alkyloxazoline macromonomers as are described, for example, in EP 408 311.

In addition, it is also possible to use fluorine-containing monomers, as are described, for example, in EP-A 558 423, or compounds which have a crosslinking action or regulate the molecular weight, in combination or on their own.

The further copolymerizable monomers b) are preferably used in an amount of 0-40% by weight, preferably 0-25% by weight, particularly preferably 0-15%.

Regulator e)

In one embodiment of the invention, the graft polymers can be obtained by carrying out the free-radical polymerization in the presence of at least one regulator e).

Regulators e) which can be used are the customary compounds known to the person skilled in the art, such as, for example, sulfur compounds (e.g.: mercaptoethanol, 2-ethylhexyl thioglycolate, thioglycolic acid or dodecylmercaptan), and tribromochloromethane or other compounds which have a regulating effect on the molecular weight of the resulting polymers.

Where appropriate, it is also possible to use silicone compounds which contain thiol groups. Preference is given to using silicone-free regulators.

The regulator e) is preferably used in an amount of 0-5% by weight, preferably 0-2.5% by weight, particularly preferably 0-1.5%.

Crosslinker d)

In one preferred embodiment, the graft polymers are prepared in the presence of a crosslinker d).

Monomers d), which have a crosslinking function, are compounds with at least 2 ethylenically unsaturated, nonconjugated double bonds in the molecule.

Suitable crosslinkers d) are, for example, acrylic esters, methacrylic esters, allyl ethers or vinyl ethers of at least dihydric alcohols. The OH groups of the parent alcohols may be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, mononeopentylglycol hydroxypivalate, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and also polyethylene glycols, polypropylene glycols and polytetrahydrofurans with molecular weights of in each case 200 to 10000. Apart from the homopolymers of ethylene oxide or propylene oxide, it is also possible to use block copolymers of ethylene oxide or propylene oxide or copolymers which comprise ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. It is of course also possible to use the polyhydric alcohols also following reaction with ethylene oxide or propylene oxide in the form of the corresponding ethoxylates or propoxylates, respectively. The polyhydric alcohols can also firstly be converted into the corresponding glycidyl ethers by reaction with epichlorohydrin.

Further suitable crosslinkers are the vinyl esters or the esters of monohydric, unsaturated alcohols with ethylenically unsaturated C₃— to C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. It is, however, also possible to esterify the monohydric, unsaturated alcohols with polyhydric carboxylic acids, for example malonic acid, tartaric acid, trimellitic acid, phthalic acid, terephthalic acid, citric acid or succinic acid.

Further suitable crosslinkers are esters of unsaturated carboxylic acids with the above-described polyhydric alcohols, for example of oleic acid, crotonic acid, cinnamic acid or 10-undecenoic acid.

Further suitable crosslinkers d) are straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1 9-decadiene, 4-vinyl-1-cyclohexene, trivinylcyclohexane or polybutadienes with molecular weights of from 200 to 20 000.

Further suitable crosslinkers are the acrylamides, methacrylamides and N-allylamines of at least difunctional amines. Such amines are, for example, diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

Triallylamine and triallylmonoalkylammonium salts, e.g. triallylmethylammonium chloride or methylsulfate, are also suitable as crosslinkers.

Also suitable are N-vinyl compounds of urea derivatives, at least difunctional amides, cyanurates or urethanes, for example of urea, ethyleneurea, propyleneurea or tartaramide, e.g. N,N′-divinylethyleneurea or N,N′-divinylpropyleneurea.

Further suitable crosslinkers are divinyldioxane, tetraallylsilane or tetravinylsilane.

It is of course also possible to use mixtures of the abovementioned compounds. Preference is given to using those crosslinkers which are soluble in the monomer mixture.

Particularly preferably used crosslinkers are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers are pentaerythritol triallyl ether, methylenebisacrylamide, N,N′-divinylethyleneurea, triallylamine and triallylmonoalkylammonium salts, and acrylic esters of glycol, butanediol, trimethylolpropane or glycerol or acrylic esters of glycol, butanediol, trimethylolpropane or glycerol reacted with ethylene oxide and/or epichlorohydrin.

Polymeric Graft Base c)

The polymeric graft base c) comprises at least one compound chosen from the group c1) and at least one compound chosen from the group c2), where
c1) are polyether-containing compounds
c2) are polymers which comprise at least 5% by weight of vinylpyrrolidone units in copolymerized form.
   Graft Base c1)

Polyether-containing compounds c1) which can be used are either polyalkylene oxides based on ethylene oxide, propylene oxide, butylene oxide and further alkylene oxides, and also polyglycerol. Depending on the nature of the monomer building blocks, the polymers comprise the following structural units:
—(CH₂)₂—O—, —(CH₂)₃—O—, —(CH₂)₄—O—, —CH₂—CH(R⁹)—O—, —CH₂—CHOR¹⁰—CH₂—O—

where R⁹ is C₁-C₂₄-alkyl;

R¹⁰ is hydrogen, C_{1-C}₂₄-alkyl, R⁹—C(═O)—, R⁹—NH—C(═O)—.

The structural units may either be homopolymers or random copolymers and block copolymers.

Suitable graft bases c1) are, in particular, water-soluble polyether-containing compounds. In this connection, it is possible to use either polyalkylene oxides based on ethylene oxide, propylene oxide, butylene oxide and further alkylene oxides, as well as polyglycerol. In this connection, the structural units may either be homopolymers or random copolymers and block copolymers.

As graft base c1), preference is given to using compounds of the following formula (I): 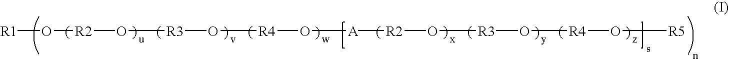
in which the variables, independently of one another, have the following meanings:

R¹ is hydrogen, C₁-C₂₄-alkyl, R⁶—C(═O)—, R⁶—NH—C(═O)—, polyalcohol radical;

R⁵ is hydrogen, C₁-C₂₄-alkyl, R⁶—C(═O)—, R⁶—NH—C(═O)—;

R²to R⁴ are —(CH₂)₂—, —(CH₂)₃—, —(CH₂)₄—, —CH₂—CH(R⁶)—, —CH₂—CHOR⁷—CH₂—;

R⁶ is C₁-C₂₄-alkyl;

R⁷ is hydrogen, C₁-C₂₄-alkyl, R⁶—C(═O)—, R⁶—NH—C(═O)—;

A is —C(═O)—O, —C(═O)—B—C(═O)—O, —CH₂—CH(—OH)—B—CH(—OH)—CH₂—O, —C(═O)— NH—B—NH—C(═O)—;

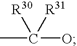

B is —(CH₂)ₜ—, arylene, optionally substituted;

R³⁰, R³¹ are hydrogen, C₁-C₂₄-alkyl, C₁-C₂₄-hydroxyalkyl, benzyl or phenyl;

n is 1 when R¹ is not a polyalcohol radical or

n is 1 to 1000 when R¹ is a polyalcohol radical

s=0 to 1000; t=1 to 12; u=1 to 5000; v=0 to 5000; w=0 to 5000;

x=0 to 5000; y=0 to 5000; z=0 to 5000.

Alkyl radicals for R⁶ and R³⁰ and R³¹ which may be mentioned are branched or unbranched C₁-C₂₄-alkyl chains, preferably methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

Preferred representatives of the abovementioned alkyl radicals which may be mentioned are branched or unbranched C₁-C₁₂—, particularly preferably C₁-C₆-alkyl chains.

As graft base c1), preference is given to polyalkylene glycols, such as, for example, polyethylene glycols and polypropylene glycols. Particular preference is given to polyethylene glycols.

The molecular weight of the polyethers c1) is in the range greater than 300 (number-average), preferably in the range from 300 to 100 000, particularly preferably in the range from 500 to 50 000, very particularly preferably in the range from 800 to 40 000.

Homopolymers of ethylene oxide or copolymers with an ethylene oxide content of from 40 to 99% by weight are used advantageously. For the ethylene oxide polymers which are preferably to be used, the content of copolymerized ethylene oxide is thus 40 to 100 mol %. Suitable comonomers for these copolymers are propylene oxide, butylene oxide and isobutylene oxide. Copolymers of ethylene oxide and propylene oxide, copolymers of ethylene oxide and butylene oxide, and copolymers of ethylene oxide, propylene oxide and at least one butylene oxide, for example, are suitable. The ethylene oxide content in the copolymers is preferably 40 to 99 mol %, the propylene oxide content is 1 to 60 mol % and the content of butylene oxide in the copolymers is 1 to 30 mol %. As well as straight-chain types, it is also possible to use branched homopolymers or copolymers as polyether-containing compounds c1).

Branched polymers can be prepared by adding ethylene oxide and optionally also propylene oxide and/or butylene oxides onto, for example, polyalcohol radicals, e.g. onto pentaerythritol, glycerol or onto sugar alcohols, such as D-sorbitol and D-mannitol, and also onto polysaccharides, such as cellulose and starch. The alkylene oxide units may be randomly distributed or be present in the form of blocks within the polymer.

It is, however, also possible to use polyesters of polyalkylene oxides and aliphatic or aromatic dicarboxylic acids, e.g. oxalic acid, succinic acid, adipic acid and terephthalic acid with molar masses of from 1500 to 25 000, as described, for example, in EP-A-0 743 962, as polyether-containing compound. Furthermore, it is also possible to use polycarbonates by reacting polyalkylene oxides with phosgene or carbonates, such as, for example, diphenyl carbonate, and also polyurethanes by reacting polyalkylene oxides with aliphatic and aromatic diisocyanates.

As polyether c1), particular preference is given to polymers of the formula III with an average molecular weight of from 300 to 100 000 (number-average), in which the variables, independently of one another, have the following meanings:

R⁴ is hydrogen, C₁-C₁₂-alkyl, R⁹—C(═O)—, R⁹—NH—C(═O)—, polyalcohol radical;

R⁸ is hydrogen, C₁-C₁₂-alkyl, R⁹—C(═O)—, R⁹—NH—C(═O)—;

R⁵ to R⁷ are —(CH₂)₂—, —(CH₂)₃—, —(CH₂)₄—, —CH₂—CH(R⁹)—, —CH₂—CHOR¹⁰—CH₂—;

R⁹ is C₁-C₁₂-alkyl;

R¹⁰ is hydrogen, C₁-C12-alkyl, R⁹—C(═O)—, R⁹—NH—C(═O)—;

n=1 to 8; s=0; u=2 to 2000; v=0 to 2000; w=0 to 2000.

As polyether c1), very particular preference is given to polymers of the formula III with an average molecular weight of from 500 to 50 000 (number-average), in which the variables, independently of one another, have the following meanings:

R⁴ is hydrogen, C₁-C₆-alkyl, R⁹—C(═O)—, R⁹—NH—C(═O)—;

R⁸ is hydrogen, C₁-C₆-alkyl, R⁹—C(═O)—, R⁹—NH—C(═O)—;

R⁵ to R⁷ are —(CH₂)₂—, —(CH₂)₃—, —(CH₂)₄—, —CH₂—CH(R⁹)—, —CH₂—CHOR¹⁰—CH₂—;

R⁹ is C₁-C₆-alkyl;

R¹⁰ is hydrogen, C₁-C₆-alkyl, R⁹—C(═O)—, R⁹—NH—C(═O)—;

n=1; s=0; u=5 to 500; v=0 to 500; w=0 to 500.

However, the polyethers c1) used may also be silicone derivatives. Suitable silicone derivatives are the compounds known under the INCI name dimethicone copolyols or silicone surfactants, such as, for example, those available under the trade names Abil™ (T. Goldschmidt), Alkasil™ (Rhône-Poulenc), Silicone Polyol Copolymer™ (Genesee), Belsil™ (Wacker), Silwet™ (Witco, Greenwich, Conn., USA) or Dow Corning (Dow Corning). These include compounds with the CAS numbers 64365-23-7; 68937-54-2; 68938-54-5; 68937-55-3.

Silicones are generally used in hair cosmetics to improve the feel. The use of polyether-containing silicone derivatives as polyethers c1) in the polymers according to the invention can therefore additionally lead to an improvement in the feel of the hair.

Preferred representatives of such polyether-containing silicone derivatives are those which comprise the following structural elements: 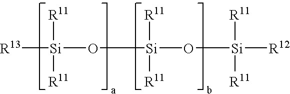
where: 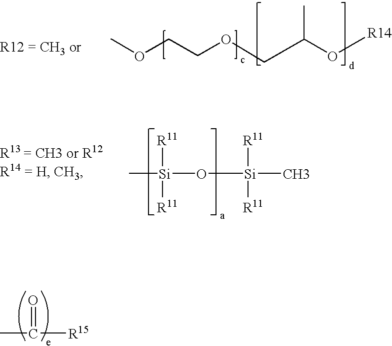
R¹⁵ is an organic radical containing 1 to 40 carbon atoms and which can comprise amino, carboxylic acid or sulfonate groups, or for the case e=0, is also the anion of an inorganic acid,
   and where the radicals R¹¹ may be identical or different, and either originate from the group of aliphatic hydrocarbons having 1 to 20 carbon atoms, are cyclic aliphatic hydrocarbons having 3 to 20 carbon atoms, are of an aromatic nature or are identical to R¹², where: 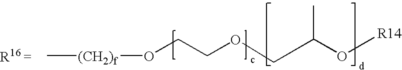
   with the proviso that at least one of the radicals R¹¹, R¹² or R¹³ is a polyalkylene oxide-containing radical according to the abovementioned definition and f is an integer from 1 to 6, a and b are integers such that the molecular weight of the polysiloxane block is between 300 and 30 000, c and d may be integers between 0 and 50, with the proviso that the sum of c and d is greater than 0, and e is 0 or 1.

Preferred radicals R¹² and R¹⁶ are those in which the sum c+d is between 5 and 30.

The groups R¹¹ are preferably chosen from the following group: methyl, ethyl, propyl, butyl, isobutyl, pentyl, isopentyl, hexyl, octyl, decyl, dodecyl and octadecyl, cycloaliphatic radicals, specifically cyclohexyl, aromatic groups, specifically phenyl or naphthyl, mixed aromatic-aliphatic radicals such as benzyl or phenylethyl and tolyl and xylyl and R¹⁶.

Particularly suitable radicals R¹⁴ are those in which in the case where R¹⁴═—(CO)ₑ—R¹⁵, R¹⁵ is a desired alkyl, cycloalkyl or aryl radical which has between 1 and 40 carbon atoms and which can carry further ionogenic groups such as NH₂, COOH, SO₃H.

Preferred inorganic radicals R¹⁵ are, for the case e=0, phosphate and sulfate.

Particularly preferred polyether-containing silicone derivatives c-1) are those of the structure:

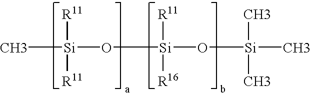

In addition, homo- and copolymers of polyalkylene oxide-containing ethylenically unsaturated monomers, such as, for example, polyalkylene oxide(meth)acrylates, polyalkylene oxide vinyl ethers, polyalkylene oxide(meth)acrylamides, polyalkylene oxide allylamides or polyalkylene oxide vinylamides can also be used as polyethers (c1). It is of course also possible to use copolymers of such monomers with other ethylenically unsaturated monomers.

As polyether-containing compounds c1), it is, however, also possible to use reaction products of polyethyleneimines with alkylene oxides. In this case, the alkylene oxides used are preferably ethylene oxide, propylene oxide, butylene oxide and mixtures thereof, particularly preferably ethylene oxide. Polyethyleneimines which can be used are polymers having number-average molecular weights of from 300 to 20 000, preferably from 500 to 10 000, very particularly preferably from 500 to 5 000. The weight ratio between used alkylene oxide and polyethyleneimine is in the range from 100:1 to 0.1:1, preferably in the range from 50:1 to 0.5:1, very particularly preferably in the range from 20:1 to 0.5:1.

Graft Base c2)

As well as at least one compound from the group of polyether-containing compounds c1), the graft base c) comprises at least one compound from the group of polymers c2) which comprise at least 5% by weight of vinylpyrrolidone units.

Preferably, these polymers used as graft base comprise a vinylpyrrolidone content of at least 10% by weight, very particularly preferably of at least 30% by weight, in particular at least 50% by weight, preferably at least 80% by weight.

Particularly preferred graft bases c) are polyvinylpyrrolidone homopolymers.

Suitable comonomers of the vinylpyrrolidone for the synthesis of the graft base (c2) are, for example, N-vinylcaprolactam, N-vinylimidazole, N-vinyl-2-methylimidazole, N-vinyl-4-methylimidazole, 3-methyl-1-vinylimidazolium chloride, 3-methyl-1-vinylimidazolium methyl sulfate, diallyldimethylammonium chloride, styrene, alkylstyrenes.

Further suitable comonomers for the preparation of the graft base c2) are, for example, monoethylenically unsaturated C₃-C₆-carboxylic acids, such as, for example, acrylic acid, methacrylic acid, crotonic acid, fumaric acid, and esters, amides and nitriles thereof, such as, for example, methyl acrylate, ethyl acrylate, methyl methacrylate, ethyl methacrylate, stearyl methacrylate, hydroxyethyl acrylate, hydroxypropyl acrylate, hydroxybutyl acrylate, hydroxyethyl methacrylate, hydroxypropyl methacrylate, hydroxyisobutyl acrylate, hydroxyisobutyl methacrylate, monomethyl maleate, dimethyl maleate, monoethyl maleate, diethyl maleate, 2-ethylhexyl acrylate, 2-ethylhexyl methacrylate, maleic anhydride and monoesters thereof, alkylene glycol(meth)acrylates, acrylamide, methacrylamide, N,N-dimethylacrylamide, N-tert-butylacrylamide, acrylonitrile, methacrylonitrile, vinyl ethers, such as, for example, methyl, ethyl, butyl or dodecyl vinyl ethers, cationic monomers, such as dialkylaminoalkyl(meth)acrylates and dialkylaminoalkyl(meth)acrylamides, such as dimethylaminomethyl acrylate, diethylaminoethyl acrylate, diethylaminoethyl methacrylate, and the salts of the last-named monomers with carboxylic acids or mineral acids, and the quaternized products.

The graft base c2) is prepared by known processes, for example, solution, precipitation, suspension or emulsion polymerization using compounds which form free radicals under the polymerization conditions. The polymerization temperatures are usually in the range from 30 to 200, preferably 40 to 110° C. Suitable initiators are, for example, azo and peroxy compounds, and the customary redox initiator systems, such as combinations of hydrogen peroxide and compounds which have a reducing action, for example sodium sulfite, sodium bisulfite, sodium formaldehyde sulfoxilate and hydrazine. These systems may also additionally comprise small amounts of a heavy metal salt.

The homopolymers and copolymers (graft base C2) have K values of at least 7, preferably 10 to 250. However, the polymers may have K values up to 300. The K values are determined in accordance with H. Fikentscher, Cellulose-Chemie, Volume 13, 58 to 64 and 71 to 74 (1932) in an aqueous solution at 25° C., at concentrations between 0.1% and 5% depending on the K value range.

Component c) (sum of components c1 and c2) is preferably used in an amount of from 10 to 90% by weight, in particular 20 to 70% by weight, preferably 30 to 60% by weight.

In a preferred embodiment of the invention, a mixture comprising at least one compound c1) polyalkylene glycols and at least one compound c2) polymers which at least 50% by weight of vinylpyrrolidone units is used as graft base c).

In a preferred embodiment of the invention, a mixture comprising at least one compound c1) polyalkylene glycols and at least one compound c2) polymers which at least 50% by weight of vinylpyrrolidone units is used as graft base c), and the polymerization is carried out in the presence of a crosslinker d).

In a preferred embodiment of the invention, a mixture comprising at least one compound c1) polyalkylene glycols and at least one compound c2) polymers which at least 80% by weight of vinylpyrrolidone units is used as graft base c).

In a preferred embodiment of the invention, a mixture comprising at least one compound c1) polyalkylene glycols and at least one compound c2) polymers which at least 80% by weight of vinylpyrrolidone units is used as graft base c), and the polymerization is carried out in the presence of a crosslinker d).

In a preferred embodiment of the invention, a mixture comprising at least one compound c1) polyalkylene glycols and at least one compound c2) polyvinylpyrrolidone homopolymer is used as graft base c).

In a preferred embodiment of the invention, a mixture comprising at least one compound c1) polyalkylene glycols and at least one compound c2) polyvinylpyrrolidone homopolymer is used as graft base c), and the polymerization is carried out in the presence of a crosslinker d).

Component d) is preferably used in an amount of from 0 to 10% by weight, in particular from 0.01 to 10% by weight, in particular 0.05 to 5% by weight, preferably 0.1 to 1.5% by weight.

Preparation of the Graft Polymers

It is of course also possible to copolymerize mixtures of the respective monomers from group a), such as, for example, mixtures of N-vinylformamide and N-vinylacetamide.

Component a) is preferably used in an amount of from 10 to 90% by weight, in particular 20 to 70% by weight, preferably 30 to 60% by weight. In a preferred embodiment, the components a), c) and d) are used in the following amounts. The individual percentages by weight given here always refer to the total sum of components a), c) and d), which is set as 100%. If further possible components are present (e.g. components b) and e), then the weights given of these further components are calculated on the basis of the sum of a) to d), which is set as 100%.

Component a) is preferably used in an amount of from 10 to 90% by weight, in particular 20 to 70% by weight, preferably 30 to 60% by weight.

Component c) is preferably used in in an amount of from 90 to 10% by weight, in particular 70 to 20% by weight, preferably 60 to 30% by weight.

Component d) is preferably used in an amount of from 0 to 10% by weight, in particular 0.01 to 10% by weight, in particular 0.05 to 5% by weight, preferably 0.1 to 1.5% by weight.

Particular preference is given to graft polymers obtainable by free-radical polymerization of

10 to 90% by weight, in particular 20 to 70% by weight, preferably 30 to 60% by weight, of component a)

90 to 10% by weight, in particular 70 to 20% by weight, preferably 60 to 30% by weight, of component c)

0 to 10% by weight, preferably 0,01 to 10% by weight, in particular 0.05 to 5% by weight, preferably 0.1 to 1.5% by weight, of component d)

with the proviso that the sum of a), c) and d) adds up to 100%.

If further components are present, these are preferably present in the following amounts (based on the 100% of the sum of a), c) and d))
0-60% by weight, preferably 0 to 40% by weight, preferably 0-25% by weight, particularly preferably 0-15% by weight, of component b)
0-5% by weight, preferably 0-2.5% by weight, particularly preferably 0-1.5% by weight, of component e).

Preference is given to graft polymers obtainable by free-radical graft copolymerization of
a) 10-90% by weight, in particular 20 to 80% by weight, of at least one open-chain N-vinylamide compound of the formula I and
b) 0-60% by weight, in particular 0-25% by weight of one or more further copolymerizable monomers onto
c) 90-10% by weight, in particular 80 to 20% by weight, of a water-soluble or water-dispersible polymeric graft base c).

Preference is given to graft polymers obtainable by free-radical graft copolymerization of
a) 10-90% by weight, in particular 20 to 80% by weight, of at least one open-chain N-vinylamide compound of the formula I and
b) 0-60% by weight, in particular 0-25% by weight, of one or more further copolymerizable monomers onto
c) 90-10% by weight, in particular 80 to 20% by weight, of a water-soluble or water-dispersible polymeric graft base c)
d) 0-10% by weight, in particular 0.01-10% by weight, of a crosslinker d).

Very particular preference is given to graft polymers obtainable by free-radical graft copolymerization of
a) 30-60% by weight of at least one open-chain N-vinyl-amide compound of the formula I and
b) 0-40% by weight of one or more further copolymerisable monomers onto
c) 60-30% by weight of one or more water-soluble or water-dispersible polymeric graft base c)
d) 0.05-5% by weight of a crosslinker d).

To prepare the polymers, the monomers of component a) and optionally of component b) may be polymerized in the presence of the graft base c)[=c1) and c2)] either using initiators which form free radicals, or by the action of high-energy radiation, which is also intended to mean the action of high-energy electrons.

Initiators which can be used for the free-radical polymerization are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butylperoxy-2-ethyl hexanoate, tert-butyl permaleate, cumene hydroperoxide, diisopropyl peroxydicarbamate, bis(o-toloyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, azobis(2-amidinopropane) dihydrochloride or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate.

Preference is given to using organic peroxides.

The polymerization can also be carried out by the action of ultraviolet radiation, optionally in the presence of UV initiators. For the polymerization under the action of UV rays, use is made of the suitable photoinitiators and/or or sensitizers customary for this purpose. These are, for example, compounds such as benzoin and benzoin ether, α-methylbenzoin or α-phenylbenzoin. It is also possible to use “triplet sensitizers”, such as benzyl diketals. The UV radiation sources used are, for example, in addition to high-energy UV lamps, such as carbon arc lamps, mercury vapor lamps or xenon lamps, also low-UV light sources, such as fluorescent tubes with a high blue component.

The amounts of initiator or initiator mixtures used, based on monomer used, are between 0.01 and 10% by weight, preferably between 0.1 and 5% by weight.

The polymerization is carried out in the temperature range from 40 to 200° C., preferably in the range from 50 to 140° C., particularly preferably in the range from 60 to 110° C. It is usually carried out under atmospheric pressure, but can also be carried out under reduced or increased pressure, preferably between 1 and 5 bar.

The polymerization can, for example, be carried out as solution polymerization, bulk polymerization, emulsion polymerization, inverse emulsion polymerization, suspension polymerization, inverse suspension polymerization or precipitation polymerization, without the possible methods being limited thereto.

In the case of bulk polymerization, the procedure may involve dissolving the graft base c) in at least one monomer of group a) and possibly other comonomers of group b) and, after the addition of a polymerization initiator, fully polymerizing the mixture. The polymerization can also be carried out semicontinuously by firstly introducing some, e.g. 10%, of the mixture to be polymerized comprising the graft base c), at least one monomer of group a), possibly other comonomers of group b) and initiator, heating the mixture to the polymerization temperature and, after the polymerization has started, adding the remainder of the mixture to be polymerized in accordance with the progress of the polymerization. The polymers can also be obtained by initially introducing the graft base c) into a reactor, heating it to the polymerization temperature and adding at least one monomer of group a), possibly other comonomers of group b) and polymerization initiator either in one portion, step by step or, preferably, continuously, and polymerizing.

If desired, the above-described polymerization can also be carried out in a solvent. Suitable solvents are, for example, alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of dihydric alcohols, diethylene glycol, triethylene glycol, glycerol and dioxane. The polymerization can also be carried out in water as solvent. In this case, the initial charge is a solution which, depending on the amount of monomers of component a) added, is soluble in water to a greater or lesser degree. In order to convert water-insoluble products, which can form during the polymerization, into solution, it is possible, for example, to add organic solvents, such as monohydric alcohols having from 1 to 3 carbon atoms, acetone or dimethylformamide. However, in the case of polymerization in water, it is also possible to convert the water-insoluble polymers into a finely divided dispersion by addition of customary emulsifiers or protective colloids, e.g. polyvinyl alcohol.

The emulsifiers used are, for example, ionic or nonionic surfactants whose HLB value is in the range from 3 to 13. The definition of the HLB value can be found in the publication by W. C. Griffin, J. Soc. Cosmetic Chem., Volume 5, 249 (1954).

The amount of surfactants, based on the graft polymer, is 0.1 to 10% by weight. Using water as solvent gives solutions or dispersions of the polymers. If solutions of the polymer are prepared in an organic solvent or in mixtures of an organic solvent and water, then, per 100 parts by weight of the polymer, 5 to 2 000, preferably 10 to 500, parts by weight of the organic solvent or of the solvent mixture are used.

The graft copolymers according to the invention can be hydrolyzed after the polymerization. The hydrolysis produces a cationic group in the polymer. This may lead to increased solubility in water and improved conditioning properties in cosmetic applications.

From the above-described graft copolymers arise, by partial or complete elimination of the formyl groups or of the C₁-C₆-alkyl-C═O— groups from those open-chain N-vinylamides (IV) incorporated into the polymer, with the formation of amine and/or ammonium groups, units of the formula (V)

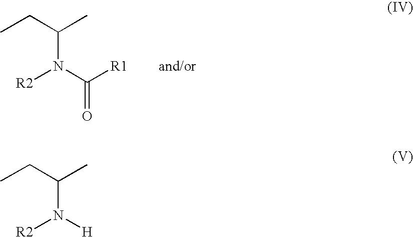

In the formulae (IV) and (V), the substituents R¹ and R² are each as defined above. Depending on the reaction conditions chosen during the hydrolysis, either partial or complete hydrolysis of the units (IV) is achieved.

If, in addition to the hydrolysis-insensitive vinylpyrrolidone units, the graft base also comprises comonomers which are hydrolysis-sensitive, such as, for example, vinyl acetate or acrylamide, then hydrolysis also takes place in the graft base. Thus, vinyl acetate reacts to give vinyl alcohol groups, and acrylamide reacts to give acrylic acid groups.

Suitable hydrolysis agents are mineral acids, such as hydrogen halides, which can be used in gaseous form or in aqueous solution. Preference is given to using hydrochloric acid, sulfuric acid, nitric acid and phosphoric acid, and organic acids, such as C₁-C₅-carboxylic acids and aliphatic or aromatic sulfonic acids. 0.05 to 2 mol equivalents, preferably 1 to 1.5 mol equivalents, of an acid are required per formyl group equivalent which is to be eliminated from the copolymerized units (IV).

The hydrolysis of the copolymerized units of the structure (IV) can also be carried out using bases, e.g. metal hydroxides, in particular alkali metal and alkaline earth metal hydroxides. Preference is given to using sodium hydroxide or potassium hydroxide. The hydrolysis can optionally also be carried out in the presence of ammonia or amines.

The hydrolysis in the acidic or in the alkaline pH range takes place, for example, at temperatures of from 30 to 170, preferably 50 to 120° C. It is complete after about 2 to 8 hours, preferably 3 to 5 hours. After these reaction times, degrees of hydrolysis of the units of the copolymerized monomers of the formula (I) of from 1 to 100% are achieved. A particularly successful procedure has proven to be one in which the bases or acids are added in aqueous solution for the hydrolysis. After the hydrolysis, a neutralization is generally carried out, such that the pH of the hydrolyzed polymer solution is 2 to 8, preferably 3 to 7. Neutralization is required if a continuation of the hydrolysis of partially hydrolyzed polymers is to be avoided or delayed. The hydrolysis can also be carried out using enzymes.

The polymers prepared in this way can then be cationized by reaction of hydroxyl and/or amino functions present in the polymer with epoxides of the formula X(R³¹═C₁ to C₄₀ alkyl).

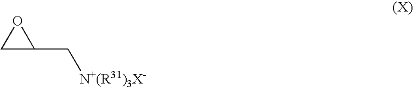

For this, the hydroxyl groups of the polyvinyl alcohol units and vinylamine units, formed by hydrolysis of vinylformamide, can preferably be reacted with the epoxides. The epoxides of the formula X can also be produced in situ by reaction of the corresponding chlorohydrins with bases, for example sodium hydroxide.

Preference is given to using 2,3-epoxypropyltrimethylammonium chloride or 3-chloro-2-hydroxypropyltrimethylammonium chloride.

The K values of the polymers should be in the range from 10 to 300, preferably 25 to 250, particularly preferably 25 to 200, very particularly preferably in the range from 30 and 150. The K value desired in each case can be adjusted in a manner known per se through the composition of the feed substances. The K values are determined in accordance with Fikentscher, Cellulosechemie, Vol. 13, pp. 58 to 64, and 71 to 74 (1932) in N-methylpyrrolidone at 25° C. and polymer concentrations which, depending on the K value range, are between 0.1% by weight and 5% by weight.

To remove solvents, the polymer solutions can be steam-distilled. Following steam distillation, aqueous solutions or dispersions are obtained depending on the choice of components a-c.

The graft polymers obtained can also be subsequently crosslinked by reacting the hydroxyl groups or amino groups in the polymer with at least bifunctional reagents. In the case of low degrees of crosslinking, water-soluble products are obtained, and in the case of high degrees of crosslinking, water-swellable or insoluble products are obtained.

For example, the polymers according to the invention can be reacted with dialdehydes and diketones, e.g. glyoxal, glutaraldehyde, succindialdehyde or terephthalaldehyde. Also suitable are aliphatic or aromatic carboxylic acids, for example maleic acid, oxalic acid, malonic acid, succinic acid or citric acid, or carboxylic acid derivatives, such as carboxylic esters, anhydrides or halides. Also suitable are polyfunctional epoxides, e.g. epichlorohydrin, glycidyl methacrylate, ethylene glycol diglycidyl ether, 1,4-butanediol diglycidyl ether or 1,4-bis(glycidyloxy)benzene. Also suitable are diisocyanates, for example hexamethylene diisocyanate, isophorone diisocyanate, methylenediphenyl diisocyanate, toluylene diisocyanate or divinylsulfone.

Also suitable are inorganic compounds, such as boric acid or boric acid salts, for example sodium metaborate, borax (disodium tetraborate), and salts of polyvalent cations, e.g. copper(II) salts, such as copper(II) acetate, or zinc, aluminum, titanium salts.

Boric acid and/or boric acid salts, such as sodium metaborate or disodium tetraborate, are preferably suitable for the subsequent crosslinking. In this connection, boric acid and/or boric acid salts can, preferably as salt solutions, be added to the solutions of the polymers according to the invention. Preference is given to adding boric acid and/or boric acid salts to the aqueous polymer solutions.

The boric acid and/or boric acid salts can be added to the polymer solutions directly after preparation. It is, however, also possible to add the boric acid and/or boric acid salts subsequently to the cosmetic formulations containing the polymers according to the invention, or to add them during the preparation process of the cosmetic formulations. The proportion of boric acid and/or boric acid salts, based on the polymers according to the invention, is 0 to 15% by weight, preferably 0 to 10% by weight, particularly preferably 0 to 5% by weight.

The graft polymer solutions and dispersions can be converted into powder form by a variety of drying methods, such as, for example, spray drying, fluidized spray drying, drum drying or freeze drying. The drying method used with preference is spray drying. The dry polymer powder obtained in this way can be used to prepare an aqueous solution or dispersion again, by dissolution or redispersion in water. Conversion into powder form has the advantage of better storability, easier transportation, and a lower propensity for microbial attack.

The graft copolymers according to the invention are highly suitable for use in cosmetic formulations. They are suitable in particular as conditioning agents and as thickeners.

The graft polymers according to the invention are suitable as styling agents and/or conditioning agents in hair cosmetic preparations, such as hair cures, hair lotions, hair rinses, hair emulsions, split-end fluids, neutralizers for permanent waves, “hot-oil treatment” preparations, conditioners, setting lotions or hairsprays. Depending on the field of application, the hair cosmetic preparations can be applied as spray, foam, gel, gel spray or mousse.

The hair cosmetic formulations according to the invention comprise, in a preferred embodiment
a) 0.01-20% by weight of the graft polymer according to the invention
b) 20-99.99% by weight of water and/or alcohol
c) 0-79.5% by weight of further constituents.

Alcohol is to be understood as meaning all alcohols customary in cosmetics, e.g. ethanol, isopropanol, n-propanol.

Further constituents are to be understood as meaning the additives customary in cosmetics, for example propellants, antifoams, interface-active compounds, i.e. surfactants, emulsifiers, foam formers and solubilizers. The interface-active compounds used can be anionic, cationic, amphoteric or neutral. Further customary constituents can also be, for example, preservatives, perfume oils, opacifiers, active ingredients, UV filters, care substances such as panthenol, collagen, vitamins, protein hydrolyzates, alpha- and beta-hydroxycarboxylic acids, stabilizers, pH regulators, dyes, viscosity regulators, gel formers, salts, moisturizers, refatting agents and further customary additives.

These also include all styling and conditioning polymers known in cosmetics which can be used in combination with the polymers according to the invention, in cases where very specific properties are to be set.

Suitable traditional hair cosmetic polymers are, for example, anionic polymers. Such anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes (Luviset™ P.U.R.) and polyureas. Particularly suitable polymers are copolymers of t-butyl acrylate, ethyl acrylate, methacrylic acid (e.g. Luvimer™ 100P), copolymers of N-tert-butylacrylamide, ethyl acrylate, acrylic acid (Ultrahold™ 8, strong), copolymers of vinyl acetate, crotonic acid and optionally other vinyl esters (e.g. Luviset™ grades), maleic anhydride copolymers, optionally reacted with alcohols, anionic polysiloxanes, e.g. carboxy-functional ones, copolymers of vinylpyrrolidone, t-butyl acrylate, methacrylic acid (e.g. Luviskol™ VBM).

Very particularly preferred anionic polymers are acrylates with an acid number greater than or equal to 120 and copolymers of t-butyl acrylate, ethyl acrylate or methacrylic acid.

Other suitable hair cosmetic polymers are cationic polymers with the name polyquaternium according to INCI, e.g. copolymers of vinylpyrrolidone/N-vinylimidazolium salts (Luviquat™ FC, Luviquat™ HM, Luviquat™ MS, Luviquat™ Care), copolymers of N-vinylpyrrolidone/dimethylaminoethyl methacrylate, quaternized with diethyl sulfate (Luviquat™ PQ 11), copolymers of N-vinylcaprolactam-N-vinylpyrrolidone/N-vinylimidazolium salts (Luviquat™ Hold); cationic cellulose derivatives (polyquaternium-4 and -10), acrylamide copolymers (polyquaternium-7).

Other suitable hair cosmetic polymers are also neutral polymers such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

To establish certain properties, the preparations can also additionally comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino-functional silicone compounds such as amodimethicones (CTFA).

The graft polymers according to the invention are suitable in particular as setting agents in hairstyling preparations, in particular hairsprays (aerosol sprays and pump sprays without propellant gas) and hair foams (aerosol foams and pump foams without propellant gas).

In a preferred embodiment, these preparations comprise
a) 0,1-10% by weight of the graft polymer according to the invention
b) 20-99.9% by weight of water and/or alcohol
c) 0-70% by weight of a propellant
d) 0-20% by weight of further constituents

Propellants are the propellants customarily used for hairsprays and aerosol foams. Preference is given to mixtures of propane/butane, pentane, dimethyl ether, 1,1-difluoroethane (HFC-152 a), carbon dioxide, nitrogen or compressed air.

A formulation for aerosol hair foams preferred according to the invention comprises
a) 0.1-10% by weight of the graft copolymer according to the invention
b) 55-94.8% by weight of water and/or alcohol
c) 5-20% by weight of a propellant
d) 0.1-5% by weight of an emulsifier
e) 0-10% by weight of further constituents

The emulsifiers which may be used are all emulsifiers customarily used in hair foams. Suitable emulsifiers may be nonionic, cationic or anionic.

Examples of nonionic emulsifiers (INCI nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can, for example, be chosen from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have from 1 to 10 ethylene oxide or propylene oxide units, preferably 1 to 3 ethylene oxide units, in the molecule.

A preparation suitable according to the invention for styling gels may, for example, have the following composition:
a) 0.1-10% by weight of the graft polymer according to the invention
b) 60-99.85% by weight of water and/or alcohol
c) 0.05-10% by weight of a gel former
d) 0-20% by weight of further constituents

The gel formers which can be used are all gel formers customary in cosmetics. These include slightly crosslinked polyacrylic acid, for example carbomer (INCI), cellulose derivatives, e.g. hydroxypropylcellulose, hydroxyethylcellulose, cationically modified celluloses, polysaccharides, e.g. xanthum gum, caprylic/capric triglycerides, sodium acrylates copolymer, polyquaternium-32 (and) paraffinum liquidum (INCI), sodium acrylates copolymer (and) paraffinum liquidum (and) PPG-1 trideceth-6, acrylamidopropyl trimonium chloride/acrylamide copolymer, steareth-10 allyl ether acrylates copolymer, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

The polymers according to the invention can also be used in shampoo formulations as setting and/or conditioning agents. Polymers with a cationic charge are in particular suitable as conditioning agents.

Preferred shampoo formulations comprise
a) 0.01-10% by weight of the graft polymer according to the invention
b) 25-94.99% by weight of water
c) 5-50% by. weight of surfactants
d) 0-5% by weight of a further conditioning agent
e) 0-10% by weight of further cosmetic constituents

In the shampoo formulations it is possible to use all anionic, neutral, amphoteric or cationic surfactants customarily used in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium and ammonium and triethanolamine salts. The alkyl ether sulfates, alkyl ether phosphates and alkyl ether carboxylates can have from 1 to 10 ethylene oxide or propylene oxide units, preferably 1 to 3 ethylene oxide units, in the molecule.

Suitable examples are sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates. It is possible, for example, to use cocodimethylsulfopropylbetaine, laurylbetaine, cocamidopropylbetaine or sodium cocamphopropionate.

Examples of suitable nonionic surfactants are the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkyl alkanolamides, fatty acid esters of polyethylene glycols, alkyl polyglycosides or sorbitan ether esters.

In addition, the shampoo formulations may comprise customary cationic surfactants, such as, for example, quaternary ammonium compounds, for example cetyltrimethylammonium chloride.

In the shampoo formulations it is possible to use customary conditioning agents in combination with the polymers according to the invention to achieve certain effects. These agents include, for example, cationic polymers with the INCI name polyquaternium, in particular copolymers of vinylpyrrolidone/N-vinylimidazolium salts (Luviquat™ FC, Luviquat™ HM, Luviquat™ MS, Luviquat™ Care), copolymers of N-vinylpyrrolidone/dimethylaminoethyl methacrylate, quaternized with diethyl sulfate (Luviquat™ PQ 11), copolymers of N-vinylcaprolactam/N-vinylpyrrolidone/N-vinylimidazolium salts (Luviquat™ Hold); cationic cellulose derivatives (polyquaternium-4 and -10), acrylamide copolymers (polyquaternium-7). It is also possible to use protein hydrolyzates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds such as amodimethicones (CTFA).

The graft polymers according to the invention can be prepared analogously to the following examples.

### EXAMPLES

### Synthesis Examples

### Example 1

60.0 g of polyethylene glycol having an average molecular weight of 4000 (Pluriol E 4000, BASF Aktiengesellschaft), 15 g of polyvinylpyrrolidone (K value 30), 180 g of distilled water, 2.8 g of 75% strength phosphoric acid and 2.8 g of 50% strength sodium hydroxide solution are introduced into a stirred reactor with nitrogen inlet, reflux condenser and metering device, and are refluxed under nitrogen. Under reflux, 297.1 g of vinylformamide are metered in over 1.5 hours and 10 g of tert-butyl peroctoate in 32 g of triethylene glycol monomethyl ether are metered in over 2 hours, and the mixture is further polymerized to completion at this temperature for 1.5 hours. Since the reaction mixture becomes highly viscous over the course of the reaction, 250 g of distilled water are metered in 45 minutes after the start of polymerization over the course of 1.5 hours. When the reaction is complete, the mixture is diluted with 500 g of distilled water.

### Example 2

### Hydrolysis of Example 1

500 g of the solution obtained in Example 1 are heated to 80° C. with 100 g of distilled water and 1 g of sodium pyrosulfite. After the addition of 33 g of 25% strength sodium hydroxide solution, the mixture is stirred for 3 hours at 80° C. After cooling, the mixture is adjusted to pH 8 using 15 g of 38% strength hydrochloric acid.

### Example 3

120 g of polyethylene glycol having an average molecular weight of 1500 (Pluriol E 1500, BASF Aktiengesellschaft) and 43 g of polyvinylpyrrolidone (K value 90) are introduced into a stirred reactor with nitrogen inlet, reflux condenser and metering device, and melted under nitrogen. Over the course of one hour, 18.5 g of N-vinylformamide and 1.35 g of tert-butyl peroctoate in 16.1 g of triethylene glycol monomethyl ether are metered in over the course of 1.5 hours at 90° C. is afterpolymerized for one hour. During the afterpolymerization, the reaction mixture is diluted with distilled water.

### Example 4

100 g of polyethylene glycol having an average molecular weight of 9000 (Pluriol E 9000, BASF Aktiengesellschaft) and 27 g of polyvinylpyrrolidone (K value 17) are melted in a stirred reactor with nitrogen inlet, reflux condenser and metering device. 54.6 g of N-vinylformamide and 70 mg of butanediol divinyl ether are metered in over the course of one hour, and 1.88 g of tert-butyl peroctoate in 16.1 g of triethylene glycol monomethyl ether are metered in over the course of 1.5 hours at 90° C., and then the mixture is afterpolymerized for one hour at this temperature. During the afterpolymerization, the mixture is diluted with distilled water.

### Example 5

65 g of PEG-PPG block copolymer having an average molecular weight of 8000 (Lutrol F 68, BASF Aktiengesellschaft), 7 g of polyvinylpyrrolidone/vinyl acetate copolymer (Luviskol™ VA 64 BASF Aktiegesellschaft), 180 g of distilled water, 2.8 g of 75% strength phosphoric acid and 2.8 g of 50% strength sodium hydroxide solution are introduced into a stirred reactor with nitrogen inlet, reflux condenser and metering device and heated to reflux under nitrogen. Under reflux, 410 g of vinylformamide are metered in over the course of 1.5 hours, and 10 g of tert-butyl peroctoate in 32 g of triethylene glycol monomethyl ether are metered in over 2 hours and the mixture is further polymerized to completion for 1.5 hours at this temperature. Since the reaction mixture becomes highly viscous in the course of the reaction, 250 g of distilled water are metered in 45 minutes after the start of polymerization over the course of 1.5 hours. When the reaction is complete, the mixture is diluted with 500 g of distilled water.

### Example 6

Example 6 was carried out analogously to Example 5 using 72 g of alkylpolyethlene glycol with an average molecular weight of 3500 (Pluriol A 2000, BASF Aktiengesellschaft) instead of PEG-PPG block copolymer.

### Example 7

Example 7 was carried out analogously to Example 5, using 103 g of polyethylene glycol with an average molecular weight of 20 000 instead of PEG-PPG block copolymer.

### Example 8

Example 8 was carried out analogously to Example 5 using 137 g of polyethylene glycol with an average molecular weight of 35 000 instead of PEG-PPG block copolymer.

### Example 9

Example 9 was carried out analogously to Example 5 using 103 g of polyethylene glycol with an average molecular weight of 20 000 instead of PEG-PPG block copolymer.

### Example 10

Example 10 was carried out analogously to Example 5 using 202 g of dimethicone copolyol (Belsil DMC 6031 ™, Wacker Chemie GmbH) instead of PEG-PPG block copolymer.

### Example 11

Example 11 was carried out analogously to Example 5 using 137 g of ethoxylated polyethyleneimine (prepared from 12.5% of polyethyleneimine with an average molecular weight of 1400 and 87.5% of ethylene oxide) instead of PEG-PPG block copolymer.

### Example 12

300 g of a 21.4% strength solution of polyvinylpyrrolidone having a K value of 85.0 and 140 g of polyethylene glycol with an average molecular weight of 1500 are heated to 80° C. in a gentle stream of nitrogen in a stirred reactor with nitrogen inlet, reflux condenser and metering device. Over the course of two hours are then uniformly metered in 91.7 g of N-vinylformamide and within 2.5 hours 1.83 g of 2,2′-azobis(2-amidinopropane) dihydrochloride dissolved in 98.2 g of water. When the monomer feed is complete, the reaction mixture is diluted with 239 g of water. The mixture is then afterpolymerized for 30 minutes, the temperature is increased to 85° C. and, with the addition of 0.9 g of 2,2′-azobis(2-amidinopropane)dihydrochloride, the mixture is polymerized to completion for a further hour.

### Example 13

35 g of a 30.3% strength solution of polyvinylpyrrolidone having a K value of 30, 120 g of polyethylene glycol with an average molecular weight of 6000, 451.5 g of water, 0.5 g of sodium dihydrogenphosphate and 50 g of N-vinylformamide are heated to 90° C. in a gentle stream of nitrogen in a stirred reactor with nitrogen inlet, reflux condenser and metering device. 1.0 g of 2,2′-azobis(2-amidinopropane)dihydrochloride is then added in one portion and polymerized for two hours at the reaction temperature. The temperature is then increased to 95° C. and, with the addition of 0.5 g of 2,2′-azobis(2-amidinopropane)dihydrochloride, the mixture is polymerized to completion for a further hour.

### Example 14

### Hydrolysis of Example 13

450 g of the polymer from Example 13 are heated to 80° C. Over the course of one hour, 52 g of 50% strength sodium hydroxide solution are added dropwise uniformly. The mixture is then stirred for two hours, cooled and adjusted to pH 7 with 62 g of concentrated hydrochloric acid.

### Example 15

### Hydrolysis of Example 13

450 g of the polymer from Example 13 are heated to 80° C. Over the course of one hour, 26 g of 50% strength sodium hydroxide solution are added dropwise uniformly. The mixture is then stirred for two hours, cooled and adjusted to pH 7 with 31 g of concentrated hydrochloric acid.

### Application Examples

### Example 1

### Aerosol Hair Foam Formulation

2.00% copolymer from Example 1

2.00% Luviquat Mono LS (cocotrimonium methyl sulfate)

67.7% water

10.0 propane/butane 3.5 bar (20° C.)

q.s. perfume oil

### Example 2

2.00% copolymer from Example 6

2.00% Luviquat Mono LS (cocotrimonium methyl sulfate)

67.7% water

10.0 propane/butane 3.5 bar (20° C.)

q.s. perfume oil

### Example 3

| Aerosol hair foam: | INCI |
|---|---|
| 4.00% copolymer from Example 3 | |
| 0.20% Cremophor A 25 | Ceteareth-25 |
| 1.00% Luviquat Mono CP | Hydroxyethyl Cetyldimonium |
| | Phosphate |
| 5.00% ethanol | |
| 1.00% panthenol | |
| 10.0 propane/butane 3.5 bar (20° C.) | |
| q.s. perfume oil | |
| ad 100% water | |

### Example 4

| Pump foam: | INCI |
|---|---|
| 2.00% copolymer from Example 3 | |
| 2.00% Luviflex Soft (polymer content) | |
| 1.20% 2-amino-2-methyl-1-propanol | |
| 0.20% Cremophor A 25 | |
| 0.10% Uvinul P 25 | PEG-25 PABA |
| q.s. preservative | |
| q.s. perfume oil | |
| ad 100% water | |

### Example 5

| Pump spray | INCI |
|---|---|
| 4.00% copolymer from Example 4 | |
| 1.00% panthenol | |
| 0.10% Uvinul MS 40 | Benzophenone-4 |
| q.s. preservative | |
| q.s. perfume oil | |
| ad 100% water | |

### Example 6

| Pump spray: | INCI |
|---|---|
| 4.00% copolymer from Example 3 | |
| 1.00% panthenol | |
| 0.10% Uvinul M 40 | Benzophenone-3 |
| q.s. preservative | |
| q.s. perfume oil | |
| ad 100% ethanol | |

### Example 7

| Hairspray: | INCI |
|---|---|
| 5.00% copolymer from Example 10 | |
| 0.10% silicone oil Dow Corning DC 190 | Dimethicone Copolyol |
| 35.00% dimethyl ether | |
| 5.00% n-pentane | |
| ad 100% ethanol | |
| q.s. perfume oil | |

### Example 8

| Hairspray VOC 55%: | INCI |
|---|---|
| 3.00% copolymer from Example 4 | |
| 7.00% Luviset P.U.R. | Polyurethane-1 |
| 40.00% dimethyl ether | |
| 15.00% ethanol | |
| q.s. perfume oil | |
| ad 100% water | |

### Example 9

| Hair gel: | INCI |
|---|---|
| 0.50% Carbopol 980 | Carbomer |
| 3.00% copolymer from Example 14 | |
| 0.10% phytantriol | |
| 0.50% panthenol | |
| q.s. perfume oil | |
| q.s. preservative | |
| ad 100% water | |

### Example 10

| Hair shampoo and shower gel | INCI |
|---|---|
| 0.50% copolymer from Example 3 | |
| 40.00% Texapon NSO | Sodium Laureth Sulfate |
| 5.00% Tego Betaine L 7 | Cocamidopropyl Betaine |
| 5.00% Plantacare 2000 | Decyl Glucoside |
| 1.00% propylene glycol | |
| q.s. citric acid | |
| q.s. preservative | |
| 1.00% sodium chloride | |
| ad 100% water | |

### Application Example 11

### Skin Cream

A water/oil cream emulsion (skin cream A) according to the invention was firstly prepared in accordance with the following formulation:

| Additive | INCI | % by wt. |
|---|---|---|
| Cremophor A 6 | Ceteareth-6 and Stearyl Alcohol | 2.0 |
| Cremophor A 25 | Ceteareth-25 | 2.0 |
| Lanette O | Cetearyl Alcohol | 2.0 |
| Imwitor 960 K | Glyceryl Stearate SE | 3.0 |
| Paraffin oil | | 5.0 |
| Jojoba oil | | 4.0 |
| Luvitol EHO | Cetearyl Octanoate | 3.0 |
| ABIL 350 | Dimethicone | 1.0 |
| Amerchol L 101 | Mineral Oil and Lanolin Alcohol | 3.0 |
| Veegum Ultra | Magnesium Aluminum Silicate | 0.5 |
| 1,2-Propylene glycol | Propylene Glycol | 5.0 |
| Abiol | Imidazolidinylurea | 0.3 |
| Phenoxyethanol | | 0.5 |
| D-Panthenol USP | | 1.0 |
| Polymer (Preparation | | 0.5 |
| Example 4) | | |
| Water | | ad 100 |

### Application Example 12

### Shower Gel

A shower gel formulation was prepared according to the following formulation:

| Additive | INCI | % by wt. |
|---|---|---|
| Texapon NSO | Natrium Laureth Sulfate | 40.0 |
| Tego Betaine L7 | Cocamidopropyl Betaine | 5.0 |
| Plantacare 2000 | Decyl Glucoside | 5.0 |
| Perfume | | 0.2 |
| Polymer according to | | 0.2 |
| Preparation Example 3 | | |
| Euxyl K 100 | Benzyl Alcohol, | 0.1 |
| | Methylchloroisothiazolinone, | |
| | Methylisothiazolinone | |
| D-Panthenol USP | | 0.5 |
| Citric acid (pH 6-7) | | q.s. |
| NaCl | | 2.0 |
| Water | | ad 100 |

### Application Example 13

### Humectant Formulation

Formulation A

| Additive | | INCI | % by wt. |
|---|---|---|---|
| a) | Cremophor A6 | Ceteareth-6 and Stearyl Alcohol | 2.0 |
| | Cremophor A25 | Ceteareth-25 | 2.0 |
| | Paraffin oil (high | | 10 |
| | viscosity) | | |
| | Lanette O | Cetearyl Alcohol | 2.0 |
| | Stearic acid | | 3.0 |
| | Nip-Nip | Methylparaben/Propylparaben 70:30 | 0.5 |
| | Abiol | Imidazolidinylurea | 0.5 |
| b) | Polymer | | 3.0 |
| | (Preparation | | |
| | Example 3) | | |
| | Water | | ad 100.0 |

The two phases were heated to 80° C., phase a) was stirred into b), homogenized and stirred until cold, and then the mixture was adjusted to pH 6 with 10% strength aqueous NaOH solution.

### Application Example 14

### O/W Cream for Retaining Skin Moisture

| Additive | % by wt. |
|---|---|
| Glycerol monostearate | 2.0 |
| Cetyl alcohol | 3.0 |
| Paraffin oil, subliquidum | 15.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Polymer (Preparation Example 3) | 3.0 |
| Glycerol | 3.0 |
| Sodium hydroxide | q.s. |
| Perfume oil | q.s. |
| Preservative | q.s. |
| Water | ad 100 |

### Application Example 15

### O/W Lotion

| Additive | % by wt. |
|---|---|
| Stearic acid | 1.5 |
| Sorbitan monostearate | 1.0 |
| Sorbitan monooleate | 1.0 |
| Paraffin oil, subliquidum | 7.0 |
| Cetyl alcohol | 1.0 |
| Polydimethylsiloxane | 1.5 |
| Glycerol | 3.0 |
| Polymer (Preparation Example 8) | 0.5 |
| Perfume oil | q.s. |
| Preservative | q.s. |
| Water | ad 100 |

### Application Example 16

### W/O Cream

| Additive | % by wt. |
|---|---|
| PEG-7-hydrogenated castor oil | 4.0 |
| Woolwax alcohol | 1.5 |
| Beeswax | 3.0 |
| Triglyceride, liquid | 5.0 |
| Vaseline | 9.0 |
| Ozokerite | 4.0 |
| Paraffin oil, subliquidum | 4.0 |
| Glycerol | 2.0 |
| Polymer (Preparation Example 2) | 2.0 |
| Magnesium sulfate*7H₂O | 0.7 |
| Perfume oil | q.s. |
| Preservative | q.s. |
| Water | ad 100 |

### Application Example 17

### Skincare Hydrogel

| Additive | % by wt. |
|---|---|
| Polymer (Preparation Example 10) | 3.0 |
| Sorbitol | 2.0 |
| Glycerol | 3.0 |
| Polyethylene glycol 400 | 5.0 |
| Ethanol | 1.0 |
| Perfume oil | q.s. |
| Preservative | q.s. |
| Water | ad 100 |

### Application Example 18

### Hydrodispersion Gel

| Additive | % by wt. |
|---|---|
| Polymer (Preparation Example 9) | 3.0 |
| Sorbitol | 2.0 |
| Glycerol | 3.0 |
| Polyethylene glycol 400 | 5.0 |
| Triglyceride, liquid | 2.0 |
| Ethanol | 1.0 |
| Perfume oil | q.s. |
| Preservative | q.s. |
| Water | ad 100 |

### Application Example 19

### Liquid Soap

| Additive | % by wt. |
|---|---|
| Coconut fatty acid, potassium salt | 15 |
| Potassium oleate | 3 |
| Glycerol | 5 |
| Polymer (Preparation Example 9) | 2 |
| Glycerol stearate | 1 |
| Ethylene glycol distearate | 2 |
| Specific additives, complexing agents, fragrances | q.s. |
| Water | ad 100 |

### Application Example 20

### Bodycare Cream

| Additive | INCI | % by wt. |
|---|---|---|
| Cremophor A6 | Ceteareth-6 and Stearyl | 2.0% |
| | Alcohol | |
| Cremophor A 25 | Ceteareth-25 | 2.0% |
| Grape (Vitis vinifera) | | 6.0% |
| seed oil | | |
| Glyceryl stearate SE | | 3.0% |
| Cetearyl alcohol | | 2.0% |
| Dimethicone | | 0.5% |
| Luvitol EHO | Cetearyl Octanoat | 8.0% |
| Oxynex 2004 | Propylene Glycol, BHT, Ascorbyl | 0.1% |
| | Palmitate, Glyceryl Stearate, | |
| | Citric Acid | |
| Preservative | | q.s. |
| 1,2-Propylene | | 3.0% |
| glycol USP | | |
| Glycerol | | 2.0% |
| EDTA BD | | 0.1% |
| D-Panthenol USP | | 1.0% |
| Water | | ad 100 |
| Polymer (Preparation | | 1.5% |
| Example 7) | | |
| Tocopheryl acetate | | 0.5% |

The formulation had a pH of 6.8. The viscosity (Brookfield [lacuna]

In the application examples below, all the amounts are in % by weight.

### Application Example 21

### Liquid Make-Up

A

| | |
|---|---|
| 1.70 | glyceryl stearate |
| 1.70 | cetyl alcohol |
| 1.70 | ceteareth-6 |
| 1.70 | ceteareth-25 |
| 5.20 | caprylic/capric triglyceride |
| 5.20 | mineral oil |

B

| | |
|---|---|
| q.s. | preservative |
| 4.30 | propylene glycol |
| 2.50 | polymer according to Preparation Example 3 |
| 59.50 | dist. water |

C

| | |
|---|---|
| q.s. | perfume oil |

D

| | |
|---|---|
| 2.00 | iron oxides |
| 12.00 | titanium dioxide |

Preparation:

Heat phase A and phase B separately to 80° C. Then mix phase B into phase A using a stirrer. Cool to 40° C. and add phase C and phase D. Homogenize repeatedly.

### Application Example 22

### Oil-Free Make-Up

A

| | |
|---|---|
| 0.35 | veegum |
| 5.00 | butylene glycol |
| 0.15 | xanthan gum |

B

| | |
|---|---|
| 53.0 | dist. water |
| q.s. | preservative |
| 0.2 | polysorbate-20 |
| 1.6 | tetrahydroxypropylethylenediamine |

C

| | |
|---|---|
| 1.0 | silica |
| 2.0 | nylon-12 |
| 4.15 | mica |
| 6.0 | titanium dioxide |
| 1.85 | iron oxides |

D

| | |
|---|---|
| 4.0 | stearic acid |
| 1.5 | glyceryl stearate |
| 7.0 | benzyl laurate |
| 5.0 | isoeicosane |
| q.s. | preservative |

E

| | |
|---|---|
| 1.0 | dist. water |
| 0.5 | panthenol |
| 0.1 | imidazolidinylurea |
| 5.0 | polymer according to Preparation Example 6 |

Preparation:

Wet phase A with butylene glycol, add to phase B and mix thoroughly. Heat phase AB to 75° C. Pulverize phase C feed substances, add to phase AB and homogenize thoroughly. Mix feed substances of phase D, heat to 80° C. and add to phase ABC. Mix for some time until the mixture is homogeneous. Transfer the mixture to a vessel fitted with a propeller mixer. Mix feed substances of phase E, add to phase ABCD and mix thoroughly.

### Application Example 23

### Eyeliner

A

| | |
|---|---|
| 40.6 | dist. water |
| 0.2 | disodium EDTA |
| q.s. | preservative |

B

| | |
|---|---|
| 0.6 | xanthan gum |
| 0.4 | veegum |
| 3.0 | butylene glycol |
| 0.2 | polysorbate-20 |

C

| | |
|---|---|
| 15.0 | iron oxide/Al powder/silica (e.g. Sicopearl |
| | Fantastico Gold ™ from BASF) |

D

| | |
|---|---|
| 10.0 | dist. water |
| 30.0 | polymer according to Preparation Example 9 |

Preparation:

Premix phase B. Mix phase B into phase A using a propeller mixer, allowing the thickener to swell. Wet phase C with phase D, add the mixture to phase AB and mix thoroughly.

### Application Example 24

### Shimmering Gel

A

| | |
|---|---|
| 32.6 | dist. water |
| 0.1 | disodium EDTA |
| 25.0 | carbomer (2% strength aqueous solution) |
| 0.3 | preservative |

B

| | |
|---|---|
| 0.5 | dist. water |
| 0.5 | triethanolamine |

C

| | |
|---|---|
| 10.0 | dist. water |
| 9.0 | polymer according to Preparation Example 3 |
| 1.0 | polyquaternium-46 |
| 5.0 | iron oxide |

D

| | |
|---|---|
| 15.0 | dist. water |
| 1.0 | D-panthenol 50 P (panthenol and propylene glycol) |

Preparation:

Thoroughly mix the feed substances of phase A in the order given using a propeller mixer. Then add phase B to phase A. Stir slowly until the mixture is homogeneous. Thoroughly homogenize phase C until the pigments are well distributed. Add phase C and phase D to phase AB and mix thoroughly.

### Application Example 25

### Waterproof Mascara

A

| | |
|---|---|
| 46.7 | dist. water |
| 3.0 | Lutrol E 400 (PEG-8) |
| 0.5 | xanthan gum |
| q.s. | preservative |
| 0.1 | imidazolidinylurea |
| 1.3 | tetrahydroxypropylethylenediamine |

B

| | |
|---|---|
| 8.0 | carnauba wax |
| 4.0 | beeswax |
| 4.0 | isoeicosane |
| 4.0 | polyisobutene |
| 5.0 | stearic acid |
| 1.0 | glyceryl stearate |
| q.s. | preservative |
| 2.0 | benzyl laurate |

C

| | |
|---|---|
| 10.0 | iron oxide/Al powder/silica (e.g. Sicopearl |
| | Fantastico Gold ™ from BASF) |

E

| | |
|---|---|
| 8.0 | polyurethane-1 |
| 2.0 | polymer according to Preparation Example 3 |

Preparation:

Heat phase A and phase B separately to 85° C. Maintain the temperature and add phase C to phase A and homogenize until the pigments are uniformly distributed. Add phase B to phase AC and homogenize for 2-3 minutes. Then add phase E and stir slowly. Cool the mixture to room temperature.

### Application Example 26

### Sun Protection Gel

Phase A

| | |
|---|---|
| 1.00 | PEG-40 hydrogenated castor oil |
| 8.00 | octyl methoxycinnamate (Uvinul MC 80 ™ from BASF) |
| 5.00 | octocrylene (Uvinul N 539 ™ from BASF) |
| 0.80 | octyltriazone (Uvinul T 150 ™ from BASF) |
| 2.00 | butylmethoxydibenzoylmethane (Uvinul BMBM ™ from BASF) |
| 2.00 | tocopheryl acetate |
| q.s. | perfume oil |

Phase B

| | |
|---|---|
| 2.50 | polymer according to Preparation Example 3 |
| 0.30 | acrylates/C10-30 alkyl acrylate crosspolymer |
| 0.20 | carbomer |
| 5.00 | glycerol |
| 0.20 | disodium EDTA |
| q.s. | preservative |
| 72.80 | dist. water |

Phase C

| | |
|---|---|
| 0.20 | sodium hydroxide |

Preparation:

Mix the components of phase A. Allow phase B to swell and stir into phase A with homogenization. Neutralize with phase C and homogenize again.

### Application Example 27

### Sun Protection Emulsion Containing TiO₂ and ZnO₂

Phase A

| | |
|---|---|
| 6.00 | PEG-7 hydrogenated castor oil |
| 2.00 | PEG-45/dodecyl glycol copolymer |
| 3.00 | isopropyl myristate |
| 8.00 | jojoba (Buxus chinensis) oil |
| 4.00 | octyl methoxycinnamate (Uvinul MC 80) |
| 2.00 | 4-methylbenzylidenecamphor (Uvinul MBC 95) |
| 3.00 | titanium dioxide, dimethicone |
| 1.00 | dimethicone |
| 5.00 | zinc oxide, dimethicone |

Phase B

| | |
|---|---|
| 2.00 | polymer according to Preparation Example 2 |
| 0.20 | disodium EDTA |
| 5.00 | glycerol |
| q.s. | preservative |
| 58.80 | dist. water |

Phase C

| | |
|---|---|
| q.s. | perfume oil |

Preparation:

Heat phases A and B separately to about 85° C. Stir phase B into phase A and homogenize. Cool to about 40° C., add phase C and briefly homogenize again.

### Application Example 28

### Sun Protection Lotion

Phase A

| | |
|---|---|
| 6.00 | octyl methoxycinnamate (Uvinul MC 80 ™ from BASF) |
| 2.50 | 4-methylbenzylidenecamphor (Uvinul MBC 95 ™ from BASF) |
| 1.00 | octyltriazone (Uvinul T 150 ™ from BASF) |
| 2.00 | butylmethoxydibenzoylmethane (Uvinul BMBM ™ from BASF) |
| 2.00 | PVP/hexadecene copolymer |
| 5.00 | PPG-3 myristyl ether |
| 0.50 | dimethicone |
| 0.10 | BHT, ascorbyl palmitate, citric Acid, glyceryl stearate, |
| | propylene glycol |
| 2.00 | cetyl alcohol |
| 2.00 | potassium cetyl phosphate |

Phase B

| | |
|---|---|
| 2.50 | polymer according to Preparation Example 3 |
| 5.00 | propylene glycol |
| 0.20 | disodium EDTA |
| q.s. | preservative |
| 63.92 | dist. water |

Phase C

| | |
|---|---|
| 5.00 | mineral oil |
| 0.20 | carbomer |

Phase D

| | |
|---|---|
| 0.08 | sodium hydroxide |

Phase E:

| | |
|---|---|
| q.s. | perfume oil |

Preparation:

Heat phases A and B separately to about 80° C. Stir phase B into phase A with homogenization, briefly afterhomogenize. Slurry phase C, stir into phase AB, neutralize with phase D and afterhomogenize. Cool to about 40° C., add phase E, homogenize again.

### Application Example 29

### Removable Face Mask

Phase A

| | |
|---|---|
| 57.10 | dist. water |
| 6.00 | polyvinyl alcohol |
| 5.00 | propylene glycol |

Phase B

| | |
|---|---|
| 20.00 | alcohol |
| 4.00 | PEG-32 |
| q.s | perfume oil |

Phase C

| | |
|---|---|
| 5.00 | polyquaternium-44 |
| 2.70 | polymer according to Preparation Example 3 |
| 0.20 | allantoin |

Preparation:

Heat phase A to at least 90° C. and stir until dissolved. Dissolve phase B at 50° C. and stir into phase A. At about 35° C. compensate the ethanol loss. Add phase C and stir.

### Application Example 30

### Face Mask

Phase A

| | |
|---|---|
| 3.00 | ceteareth-6 |
| 1.50 | ceteareth-25 |
| 5.00 | cetearyl alcohol |
| 6.00 | cetearyl octanoate |
| 6.00 | mineral oil |
| 0.20 | bisabolol |
| 3.00 | glyceryl stearate |

Phase B

| | |
|---|---|
| 2.00 | propylene glycol |
| 5.00 | panthenol |
| 2.80 | polymer according to Preparation Example 3 |
| q.s. | preservative |
| 65.00 | dist. water |

Phase C

| | |
|---|---|
| q.s. | perfume oil |
| 0.50 | tocopheryl acetate |

Preparation:

Heat phase A and B separately to about 80° C. Stir phase B into phase A with homogenization, briefly afterhomogenize. Cool to about 40° C., add phase C, homogenize again.

### Application Example 31

### Body Lotion Foam

Phase A

| | |
|---|---|
| 1.50 | ceteareth-25 |
| 1.50 | ceteareth-6 |
| 4.00 | cetearyl alcohol |
| 10.00 | cetearyl octanoate |
| 1.00 | dimethicone |

Phase B

| | |
|---|---|
| 3.00 | polymer according to Preparation Example 6 |
| 2.00 | panthenol |
| 2.50 | propylene glycol |
| q.s. | preservative |
| 74.50 | dist. water |

Phase C

| | |
|---|---|
| q.s. | perfume oil |

Preparation:

Heat phases A and B separately to about 80° C. Stir phase B into phase A and homogenize. Cool to about 40° C., add phase C and briefly homogenize again. Containerizing: 90% of active ingredient and 10% propane/butane at 3.5 bar (20° C.).

### Application Example 32

### Face Wash for Dry and Sensitive Skin

Phase A

| | |
|---|---|
| 2.50 | PEG-40 hydrogenated castor oil |
| q.s. | perfume oil |
| 0.40 | bisabolol |

Phase B

| | |
|---|---|
| 3.00 | glycerol |
| 1.00 | hydroxyethyl cetyldimonium phosphate |
| 5.00 | witch hazel (Hamamelis virginiana) distillate |
| 0.50 | panthenol |
| 0.50 | polymer according to Preparation Example 3 |
| q.s. | preservative |
| 87.60 | dist. water |

Preparation:

Dissolve phase A until clear. Stir phase B into phase A.

### Application Example 33

### Face Wash Paste with Peeling Effect

Phase A

| | |
|---|---|
| 70.00 | dist. water |
| 3.00 | polymer according to Preparation Example 3 |
| 1.50 | carbomer |
| q.s. | preservative |

Phase B

| | |
|---|---|
| q.s. | perfume oil |
| 7.00 | potassium cocoyl hydrolyzed protein |
| 4.00 | cocamidopropylbetaine |

Phase C

| | |
|---|---|
| 1.50 | triethanolamine |

Phase D

| | |
|---|---|
| 13.00 | polyethylene (Luwax A ™ from BASF) |

Preparation:

Allow phase A to swell. Dissolve phase B until clear. Stir phase B into phase A. Neutralize with phase C. Then stir in phase D.

### Application Example 34

### Face Soap

Phase A

| | |
|---|---|
| 25.0 | potassium cocoate |
| 20.0 | disodium cocoamphodiacetate |
| 2.0 | lauramide DEA |
| 1.0 | glycol stearate |
| 2.0 | polymer according to Preparation Example 3 |
| 50.0 | dist. water |
| q.s. | citric acid |

Phase B

| | |
|---|---|
| q.s. | preservative |
| q.s. | perfume oil |

Preparation:

Heat phase A to 70° C. with stirring until homogeneous. pH to 7.0 to 7.5 with citric acid. Cool to 50° C. and add phase B.

### Application Example 35

### Face Cleansing Milk, O/W Type

Phase A

| | |
|---|---|
| 1.50 | ceteareth-6 |
| 1.50 | ceteareth-25 |
| 2.00 | glyceryl stearate |
| 2.00 | cetyl alcohol |
| 10.00 | mineral oil |

Phase B

| | |
|---|---|
| 5.00 | propylene glycol |
| q.s. | preservative |
| 1.0 | polymer according to Preparation Example 3 |
| 66.30 | dist. water |

Phase C

| | |
|---|---|
| 0.20 | carbomer |
| 10.00 | cetearyl octanoate |

Phase D

| | |
|---|---|
| 0.40 | tetrahydroxypropylethylenediamine |

Phase E

| | |
|---|---|
| q.s. | perfume oil |
| 0.10 | bisabolol |

Preparation:

Heat phases A and B separately to about 80° C. Stir phase B into phase A with homogenization, and briefly afterhomogenize. Slurry phase C, stir into phase AB, neutralize with phase D and afterhomogenize. Cool to about 40° C., add phase E, homogenize again.

### Application Example 36

### Transparent Soap

| | |
|---|---|
| 4.20 | sodium hydroxide |
| 3.60 | dist. water |
| 2.0 | polymer according to Preparation Example 3 |
| 22.60 | propylene glycol |
| 18.70 | glycerol |
| 5.20 | cocoamide DEA |
| 10.40 | cocamine oxide |
| 4.20 | sodium lauryl sulfate |
| 7.30 | myristic acid |
| 16.60 | stearic acid |
| 5.20 | tocopherol |

Preparation:

Mix all ingredients. Melt the mixture at 85° C. until clear. Immediately pour into the mold.

### Application Example 37

### Peeling Cream, O/W Type

Phase A

| | |
|---|---|
| 3.00 | ceteareth-6 |
| 1.50 | ceteareth-25 |
| 3.00 | glyceryl stearate |
| 5.00 | cetearyl alcohol, sodium cetearyl sulfate |
| 6.00 | cetearyl octanoate |
| 6.00 | mineral oil |
| 0.20 | bisabolol |

Phase B

| | |
|---|---|
| 2.00 | propylene glycol |
| 0.10 | disodium EDTA |
| 3.00 | polymer according to Preparation Example 3 |
| q.s. | preservative |
| 59.70 | dist. water |

Phase C

| | |
|---|---|
| 0.50 | tocopheryl acetate |
| q.s. | perfume oil |

Phase D

| | |
|---|---|
| 10.00 | polyethylene |

Preparation:

Heat phases A and B separately to about 80° C. Stir phase B into phase A and homogenize. Cool to about 40° C., add phase C and briefly homogenize again. Then stir in phase D.

### Application Example 38

### Shaving Foam

| | |
|---|---|
| 6.00 | ceteareth-25 |
| 5.00 | poloxamer 407 |
| 52.00 | dist. water |
| 1.00 | triethanolamine |
| 5.00 | propylene glycol |
| 1.00 | PEG-75 lanolin oil |
| 5.00 | polymer according to Preparation Example 3 |
| q.s. | preservative |
| q.s. | perfume oil |
| 25.00 | sodium laureth sulfate |

Preparation:

Weigh everything together, then stir until dissolved. Containerizing: 90 parts of active substance and 10 parts of 25:75 propane/butane mixture.

### Application Example 39

### Aftershave balm

Phase A

| | |
|---|---|
| 0.25 | acrylates/C10-30 alkyl acrylate crosspolymer |
| 1.50 | tocopheryl acetate |
| 0.20 | bisabolol |
| 10.00 | caprylic/capric triglyceride |
| q.s. | perfume oil |
| 1.00 | PEG-40 hydrogenated castor oil |

Phase B

| | |
|---|---|
| 1.00 | panthenol |
| 15.00 | alcohol |
| 5.00 | glycerol |
| 0.05 | hydroxyethylcellulose |
| 1.92 | polymer according to Preparation Example 3 |
| 64.00 | dist. water |

Phase C

| | |
|---|---|
| 0.08 | sodium hydroxide |

Preparation:

Mix the components of phase A. Stir phase B into phase A with homogenization, then briefly afterhomogenize. Neutralize with phase C and homogenize again.

### Application Example 40

### Bodycare Cream

Phase A

| | |
|---|---|
| 2.00 | ceteareth-6 |
| 2.00 | ceteareth-25 |
| 2.00 | cetearyl alcohol |
| 3.00 | glyceryl stearate SE |
| 5.00 | mineral oil |
| 4.00 | jojoba (Buxus chinensis) oil |
| 3.00 | cetearyl octanoate |
| 1.00 | dimethicone |
| 3.00 | mineral oil, lanolin alcohol |

Phase B

| | |
|---|---|
| 5.00 | propylene glycol |
| 0.50 | veegum |
| 1.00 | panthenol |
| 1.70 | polymer according to Preparation Example 4 |
| 6.00 | polyquaternium-44 |
| q.s. | preservative |
| 60.80 | dist. water |

Phase C

| | |
|---|---|
| q.s. | perfume oil |

Preparation:

Heat phases A and B separately to about 80° C. Homogenize phase B. Stir phase B into phase A with homogenization, then briefly afterhomogenize. Cool to about 40° C., add phase C and briefly homogenize again.

### Application Example 41

### Toothpaste

Phase A

| | |
|---|---|
| 34.79 | dist. water |
| 3.00 | polymer according to Preparation Example 3 |
| 0.30 | preservative |
| 20.00 | glycerol |
| 0.76 | sodium monofluorophosphate |

Phase B

| | |
|---|---|
| 1.20 | sodium carboxymethylcellulose |

Phase C

| | |
|---|---|
| 0.80 | aroma oil |
| 0.06 | saccharin |
| 0.10 | preservative |
| 0.05 | bisabolol |
| 1.00 | panthenol |
| 0.50 | tocopheryl acetate |
| 2.80 | silica |
| 1.00 | sodium lauryl sulfate |
| 7.90 | dicalcium phosphate anhydrate |
| 25.29 | dicalcium phosphate dihydrate |
| 0.45 | titanium dioxide |

Preparation:

Dissolve phase A. Spread phase B into phase A and dissolve. Add phase C and stir under reduced pressure at RT for about 45 min.

### Application Example 42

### Mouthwash

Phase A

| | |
|---|---|
| 2.00 | aroma oil |
| 4.00 | PEG-40 hydrogenated castor oil |
| 1.00 | bisabolol |
| 30.00 | alcohol |

Phase B

| | |
|---|---|
| 0.20 | saccharin |
| 5.00 | glycerol |
| q.s. | preservative |
| 5.00 | poloxamer 407 |
| 0.5 | polymer according to Preparation Example 3 |
| 52.30 | dist. water |

Preparation:

Dissolve phase A and phase B separately until clear. Stir phase B into phase A.

### Application Example 43

### Denture Adhesive

Phase A

| | |
|---|---|
| 0.20 | bisabolol |
| 1.00 | beta-carotene |
| q.s. | aroma oil |
| 20.00 | cetearyl octanoate |
| 5.00 | silica |
| 33.80 | mineral oil |

Phase B

| | |
|---|---|
| 5.00 | polymer according to Preparation Example 3 |
| 35.00 | PVP (20% strength solution in water) |

Preparation:

Thoroughly mix phase A. Stir phase B into phase A.

### Application Example 32

### Skincare Cream, O/W Type

Phase A

| | |
|---|---|
| 8.00 | cetearyl alcohol |
| 2.00 | ceteareth-6 |
| 2.00 | ceteareth-25 |
| 10.00 | mineral oil |
| 5.00 | cetearyl octanoate |
| 5.00 | dimethicone |

Phase B

| | |
|---|---|
| 3.00 | polymer according to Preparation Example 3 |
| 2.00 | panthenol, propylene glycol |
| q.s. | preservative |
| 63.00 | dist. water |

Phase C

| | |
|---|---|
| q.s. | perfume oil |

Preparation:

Heat phase A and B separately to about 80° C. Stir phase B into phase A with homogenization, then briefly afterhomogenize. Cool to about 40° C., add phase C, homogenize again.

### Application Example 44

### Skincare Cream, W/O Type

Phase A

| | |
|---|---|
| 6.00 | PEG-7 hydrogenated castor oil |
| 8.00 | cetearyl octanoate |
| 5.00 | isopropyl myristate |
| 15.00 | mineral oil |
| 2.00 | PEG-45/dodecyl glycol copolymer |
| 0.50 | magnesium stearate |
| 0.50 | aluminum stearate |

Phase B

| | |
|---|---|
| 3.00 | glycerol |
| 3.30 | polymer according to Preparation Example 3 |
| 0.70 | magnesium sulfate |
| 2.00 | panthenol |
| q.s. | preservative |
| 48.00 | dist. water |

Phase C

| | |
|---|---|
| 1.00 | tocopherol |
| 5.00 | tocopheryl acetate |
| q.s. | perfume oil |

Preparation:

Heat phases A and B separately to about 80° C. Stir phase B into phase A and homogenize. Cool to about 40° C., add Phase C and briefly homogenize again.

### Application Example 45

### Lipcare Cream

Phase A

| | |
|---|---|
| 10.00 | cetearyl octanoate |
| 5.00 | polybutene |

Phase B

| | |
|---|---|
| 0.10 | carbomer |

Phase C

| | |
|---|---|
| 2.00 | ceteareth-6 |
| 2.00 | ceteareth-25 |
| 2.00 | glyceryl stearate |
| 2.00 | cetyl alcohol |
| 1.00 | dimethicone |
| 1.00 | benzophenone-3 |
| 0.20 | bisabolol |
| 6.00 | mineral oil |

Phase D

| | |
|---|---|
| 8.00 | polymer according to Preparation Example 3 |
| 3.00 | panthenol |
| 3.00 | propylene glycol |
| q.s. | preservative |
| 54.00 | dist. water |

Phase E

| | |
|---|---|
| 0.10 | triethanolamine |

Phase F

| | |
|---|---|
| 0.50 | tocopheryl acetate |
| 0.10 | tocopherol |
| q.s. | perfume oil |

Preparation:

Dissolve phase A until clear. Add phase B and homogenize. Add phase C and melt at 80° C. Heat phase D to 80° C. Add phase D to phase ABC and homogenize. Cool to about 40° C., add phase E and phase F, homogenize again.

### Application Example 46

### Glossy Lipstick

Phase A

| | |
|---|---|
| 5.30 | candelilla (Euphorbia cerifera) wax |
| 1.10 | beeswax |
| 1.10 | microcrystalline wax |
| 2.00 | cetyl palmitate |
| 3.30 | mineral oil |
| 2.40 | castor oil, glyceryl ricinoleate, octyldodecanol, |
| | carnauba, candelilla wax, |
| 0.40 | bisabolol |
| 16.00 | cetearyl octanoate |
| 2.00 | hydrogenated cocoglycerides |
| q.s. | preservative |
| 1.00 | polymer according to Preparation Example 3 |
| 60.10 | castor (Ricinus communis) oil |
| 0.50 | tocopheryl acetate |

Phase B:

| | |
|---|---|
| 0.80 | C.I. 14 720:1, Acid Red 14 Aluminum Lake |

Phase C:

| | |
|---|---|
| 4.00 | mica, titanium dioxide |

Preparation:

Weigh in the components of phase A and melt. Incorporate phase B until homogeneous. Add phase C and stir in. Cool to room temperature with stirring.

### Example 47

### Clear Conditioning Shampoo

A

| | |
|---|---|
| 15.00 | cocamidopropylbetaine |
| 10.00 | disodium cocoamphodiacetate |
| 5.00 | polysorbate 20 |
| 5.00 | decyl glucoside |
| q.s. | perfume |
| q.s. | preservative |
| 0.1-1.0 | graft polymer according to Example 3 |
| 2.00 | laureth-3 |
| ad 100 | aqua, demin. |
| q.s. | citric acid |

B:

| | |
|---|---|
| 3.00 | PEG-150 distearate |

Preparation:

Weigh in the components of Phase A and dissolve. Adjust the pH to 6-7. Add phase B and heat to 50° C. Allow to cool to room temperature with stirring.

### Example 48

### Shampoo

| | |
|---|---|
| 30.00 | sodium laureth sulfate |
| 6.00 | sodium cocoamphoacetate |
| 6.00 | cocamidopropylbetaine |
| 3.00 | sodium laureth sulfate, glycol |
| | distearate, cocamide MEA, laureth-10 |
| 0.1-1.0 | graft polymer according to Example 3 |
| 2.00 | dimethicone |
| q.s. | perfume/q.s. preservative/q.s. citric acid |
| 1.00 | sodium chloride |
| ad 100 | aqua, demin. |

Preparation of Examples 48 to 55:

Weigh in the components and dissolve. Adjust the pH to 6-7.

### Example 49

### Shampoo

| | |
|---|---|
| 30.00 | sodium laureth sulfate |
| 6.00 | sodium cocoamphoacetate |
| 6.00 | cocamidopropylbetaine |
| 3.00 | sodium laureth sulfate, glycol |
| | distearate, cocamide MEA, laureth-10 |
| 0.1-1.0 | graft polymer according to Example 3 |
| 2.00 | amodimethicone |
| q.s. | perfume/q.s. preservative/q.s. citric acid |
| 1.00 | sodium chloride |
| ad 100 | aqua, demin. |

### Example 50

### Shampoo

| | |
|---|---|
| 40.00 | sodium laureth sulfate |
| 10.00 | cocamidopropylbetaine |
| 3.00 | sodium laureth sulfate, glycol |
| | distearate, cocamide MEA, laureth-10 |
| 0.1-1.0 | graft polymer according to Example 3 |
| 2.00 | Dow Corning 3052 |
| q.s. | perfume/q.s. preservative/q.s. citric acid |
| 2.00 | cocamido DEA |
| ad 100 | aqua, demin. |

### Example 51

### Antidandruff Shampoo

| | |
|---|---|
| 40.00 | sodium laureth sulfate |
| 10.00 | cocamidopropylbetaine |
| 10.00 | disodium laureth sulfosuccinate |
| 2.50 | sodium laureth sulfate, glycol distearate, |
| | cocamide MEA, laureth-10 |
| 0.1-1.0 | graft polymer according to Example 3 |
| 0.50 | climbazole |
| q.s. | perfume/q.s. preservative/0.50 sodium chloride |
| ad 100 | aqua, demin. |

### Example 52

### Shampoo

| | |
|---|---|
| 25.00 | sodium laureth sulfate |
| 5.00 | cocamidopropylbetaine |
| 2.50 | sodium laureth sulfate, glycol distearate, cocamide MEA, |
| | laureth-10 |
| 0.1-1.0 | graft polymer according to Example 3 q.s. perfume |
| q.s. | preservative |
| 2.00 | cocamido DEA |
| ad 100 | aqua, demin. |

### Example 53

### Shampoo

| | |
|---|---|
| 20.00 | ammonium laureth sulfate |
| 15.00 | ammonium lauryl sulfate |
| 5.00 | cocamidopropylbetaine |
| 2.50 | sodium laureth sulfate, glycol distearate, cocamide MEA, |
| | laureth-10 |
| 0.1-1.0 | graft polymer according to Example 3 q.s. perfume |
| q.s. | preservative |
| 0.50 | sodium chloride |
| ad 100 | aqua, demin. |

### Example 54

### Clear Shower Gel

| | |
|---|---|
| 40.00 | sodium laureth sulfate |
| 5.00 | decyl glucoside |
| 5.00 | cocamidopropylbetaine |
| 0.1-1.0 | graft polymer according to Example |
| 31.00 | panthenol |
| q.s. | perfume/q.s. preservative/q.s. citric acid |
| 2.00 | sodium chloride |
| ad 100 | aqua, demin. |

### Example 55

### Shampoo

| | |
|---|---|
| 12.00 | sodium laureth sulfate |
| 1.50 | decyl glucoside |
| 2.50 | cocamidopropylbetaine |
| 5.00 | cocoglucoside glyceryl oleate |
| 2.00 | sodium laureth sulfate, glycol distearate, cocamide MEA, |
| | laureth-10 |
| 0.1-1.0 | graft polymer according to Example 3 |
| q.s. | preservative/q.s. Sunset Yellow C.I. 15 985/q.s. perfume |
| 1.00 | sodium chloride |
| ad 100 | aqua, demin. |

### Example 56

### Shampoo

A

| | |
|---|---|
| 40.00 | sodium laureth sulfate |
| 5.00 | sodium C12-15 pareth-15 sulfonate |
| 5.00 | decyl glucoside |
| q.s. | perfume |
| 0.10 | phytantriol |

B

| | |
|---|---|
| ad 100 | aqua, demin. |
| 0.1-1.0 | graft polymer according to Example 3 |
| 1.00 | panthenol |
| q.s. | preservative |
| 1.00 | laureth-3 |
| q.s. | citric acid |
| 2.00 | sodium chloride |

Preparation:

Weigh in the components of Phase A and dissolve. Adjust pH to 6-7. Add phase B and mix.

1. A graft polymer obtainable by free-radical graft polymerization of
a) at least one N-vinyl-containing monomer
b) optionally one or more further copolymerizable monomers onto a polymeric graft base c), which comprises at least one compound from the group c1) and at least one compound from the group c2), where
c1) represents polyether-containing compounds
c2) represents polymers which comprise at least 5% by weight of vinylpyrrolidone units
d) optionally at least one crosslinker.

2. A graft polymer as claimed in claim 1, wherein the graft polymer is water-soluble or water-dispersible.

3. A graft polymer as claimed in claim 1, wherein N-vinylamides and/or N-vinyllactams are used as monomer a).

4. A graft polymer as claimed in claim 1, wherein at least one open-chain N-vinylamide compound of the formula (I) 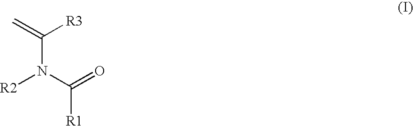
where R¹, R², R³═H or C₁— to C₆-alkyl, is used as monomer a).

5. A graft polymer as claimed in claim 4, where the radicals R¹, R² and R³ in formula (I)=H.

6. A graft polymer as claimed in claim 1, wherein N-vinyllactams of the formula (II) 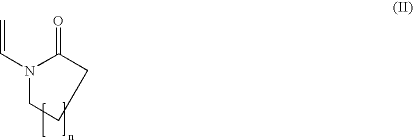
where n=1, 2, 3, are used as monomer a).

7. A graft polymer as claimed in claim 1, wherein the polyether-containing compound c1) is chosen from polymers of the formula III 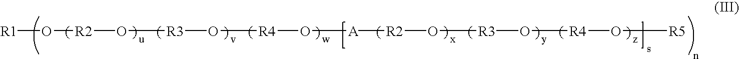
in which the variables, independently of one another, have the following meanings:
R¹ is hydrogen, C₁-C₂₄-alkyl, R⁶—C(═O)—, R⁶—NH—C(═O)—, polyalcohol radical;
R⁵ is hydrogen, C₁-C₂₄-alkyl, R⁶—C(═O)—, R⁶—NH—C(═O)—;
R² to R⁴ are —(CH₂)₂—, —(CH₂)₃—, —(CH₂)₄—, —CH₂—CH(R⁶), —CH₂—CHOR⁷—CH₂—;
R⁶ is C₁-C₂₄-alkyl;
R⁷ is hydrogen, C₁-C₂₄-alkyl, R⁶—C(═O)—, R⁶—NH—C(═O)—;
A is —C(═O)—O, —C(═O)—B—C(═O)—O, —CH₂—CH(—OH)—B—CH(—OH)—CH₂—O, —C(═O)—NH—B—NH—C(═O)—O; 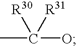
B is —CH₂)ₜ—, arylene, optionally substituted;
R³⁰, R³¹ are hydrogen, C₁-C₂₄-alkyl, C₁-C₂₄-hydroxyalkyl, benzyl or phenyl;
n is 1 when R¹ is not a polyalcohol radical or
n is 1 to 1000 when R¹ is a polyalcohol radical
s=0 to 1000; t=1 to 12; u=1 to 5000;v=0 to 5000; w=0 to 5000; x=0 to 5000; y=0 to 5000; z=0 to 5000.

8. A graft polymer as claimed in claim 1, wherein the polyether-containing compound c1) is chosen from polymers of the formula III in which the variables, independently of one another, have the following meanings:
R¹ is hydrogen, C₁-C₆-alkyl, R⁶—C(═O)—, R⁶—NH—C(═O)—;
R⁵ is hydrogen, C₁-C₆-alkyl, R⁶—C(═O)—, R⁶—NH—C(═O)—;
R² to R⁴ are —(CH₂)₂—, —(CH₂)₃—, —(CH₂)₄—, —CH₂—CH(R⁶)—, —CH₂—CHOR⁷—CH₂—;
R⁶ is C₁-C₆-alkyl;
R⁷ is hydrogen, C₁-C₆-alkyl, R⁶—C(═O)—, R⁶—NH—C(═O)—;
n=1; s=0; u=5 to 500; v=0 to 500; w=0 to 500.

9. A graft polymer as claimed in claim 1, wherein the polyether-containing compound c1) is chosen from polymers obtainable by reacting polyethylenimines with alkylene oxides.

10. A graft polymer as claimed in claim 1, wherein the polyether-containing compounds c1) have been prepared by polymerization of ethylenically unsaturated alkylene oxide-containing monomers and optionally further copolymerizable monomers.

11. A graft polymer as claimed in claim 10, wherein the polyether-containing compounds c1) have been prepared by polymerization of polyalkylene oxide vinyl ethers and optionally further copolymerizable monomers.

12. A graft polymer as claimed in claim 10, wherein the polyether-containing compounds c1) have been prepared by polymerization of polyalkylene oxide(meth)acrylates and optionally further copolymerizable monomers.

13. A graft polymer as claimed in claim 1, wherein the further comonomers of N-vinylpyrrolidone for the synthesis of the graft base c2) are chosen from the group:
N-vinylcaprolactam, N-vinylimidazole and alkyl-substituted N-vinylimidazoles, and salts thereof with carboxylic acids or mineral acids, and quaternized products thereof, unsaturated sulfonic acids, diallyldimethylammonium chloride, vinyl esters, vinyl ethers, styrene, alkylstyrenes, monoethylenically unsaturated carboxylic acids and salts, esters, amides and nitriles thereof, maleic anhydride and its monoesters, N,N-dialkylaminoalkyl(meth)acrylates, and salts thereof with carboxylic acids or mineral acids, and the quaternized products.

14. A graft polymer as claimed in claim 1, wherein a polymer with a vinylpyrrolidone content of at least 10% by weight, in particular at least 30% by weight, preferably at least 50% by weight, is used as graft base c2).

15. A graft polymer as claimed in claim 1, wherein a polyvinylpyrrolidone homopolymer is used as graft base c2).

16. A graft polymer as claimed in claim 1, wherein the graft base c used is a mixture comprising
c1) polyalkylene glycols and
c2) polymers which comprise at least 50% by weight of vinylpyrrolidone units.

17. A graft polymer as claimed in claim 1, wherein the graft base c used is a mixture comprising
c1) polyalkylene glycols and
c2) polymers which comprise at least 50% by weight of vinylpyrrolidone units
and the free-radical graft polymerization is carried out in the presence of a crosslinker d).

18. A graft polymer as claimed in claim 1, wherein the graft base c used is a mixture comprising
c1) polyalkylene glycols und
c2) polyvinylpyrrolidone homopolymers.

19. A graft polymer as claimed in claim 1, wherein the graft base c used is a mixture comprising
c1) polyalkylene glycols and
c2) polyvinylpyrrolidone homopolymers
and the free-radical graft polymerization is carried out in the presence of a crosslinker d).

20. A graft polymer as claimed in claim 1, wherein the further comonomers b) are compounds chosen from the group consisting of monoethylenically unsaturated carboxylic acids and the salts, esters, amides and nitriles of monoethylenically unsaturated carboxylic acids, maleic anhydride and its monoesters, diallyldimethylammonium chloride, vinyl esters, styrene, alkylstyrenes, unsaturated sulfonic acids, N-vinyllactams, vinyl ethers, 1-vinylimidazole and alkyl-substituted vinylimidazoles, and salts thereof with carboxylic acids or mineral acids, and quaternized products thereof, N,N-dialkylaminoalkyl (meth)acrylates, and quaternized products thereof.

21. A graft polymer as claimed in claim 1, wherein the polymer is at least partially hydrolyzed.

22. A cosmetic preparation comprising the graft polymer as claimed in claim 1.

23. The cosmetic preparation of claim 22 wherein the graft polymer acts as a thickner.

24. The cosmetic preparation of claim 22 wherein the graft polymer is used as a conditioning agent.

25. A cosmetic preparation comprising
a) 0.01-20% by weight of graft polymers as claimed in claim 1
b) 20-99.99% by weight of water and/or alcohol
c) 0-79.5% by weight of further constituents.
